SEQUENCE LISTING (6 sequences)

SEQ ID NO: 1 - DNA, 66 bp, Hepatitis C virus
  source 1..66: mol_type = other DNA
tccttctggccctgctctcttgcctgactgtgcccgcttcagcctaccaagtgcgcaattcctcgg

SEQ ID NO: 2 - DNA, 66 bp, Hepatitis C virus
  source 1..66: mol_type = other DNA
tccttttggctttgctgtcctgtttgaccatcccagcgtccgcttttgaagtgcgcaacgtgtccg

SEQ ID NO: 3 - DNA, 51 bp, Human immunodeficiency virus 1
  source 1..51: mol_type = other DNA
ccacatcccgcagggttaaaaaagaaaaaatcagtaacagtactggatgtg

SEQ ID NO: 4 - DNA, 51 bp, Human immunodeficiency virus 1
  source 1..51: mol_type = other DNA
ccacatcccgcagggttaaaaaagaacaaatcagtaacagtactggatgtg

SEQ ID NO: 5 - protein, 17 aa, Human immunodeficiency virus 1
PHPAGLKKKKSVTVLDV

SEQ ID NO: 6 - protein, 17 aa, Human immunodeficiency virus 1
PHPAGLKKNKSVTVLDV

(19) United States
(12) Patent Application Publication
(10) Pub. No.: US 2025/0179563 A1
(43) Pub. Date: Jun. 5, 2025

(54) METHODS AND SYSTEMS FOR PAMLESS DETECTION OF NUCLEIC ACIDS WITH TYPE V CRISPR/CAS SYSTEMS

(71) Applicant: University of Florida Research Foundation, Inc., Gainesville, FL (US)

(72) Inventors: Piyush K. Jain, Gainesville, FL (US); Santosh Rananaware, Gainesville, FL (US); Emma Vesco, Gainesville, FL (US); Grace M. Shoemaker, Gainesville, FL (US); Luke Samuel Sandoval, Gainesville, FL (US)

(21) Appl. No.: 18/845,013

(22) PCT Filed: Mar. 20, 2023
(86) PCT No.: PCT/US2023/064701

§ 371 Date: Sep. 9, 2024

Related U.S. Application Data
(60) Provisional application No. 63/321,408, filed on Mar. 18, 2022.

(51) Int. Cl. C12Q 1/6823; B01L 7/00; C12Q 1/44; C12Q 1/6876

(52) U.S. Cl. CPC C12Q 1/6823; B01L 7/52; C12Q 1/44; C12Q 1/6876; B01L 2300/18; G01N 2333/922

(57) ABSTRACT

Described herein are CRISPR/Cas-based methods, systems, compositions, and kits relating to the detection of one or more target polynucleotides that do not require the presence of a PAM sequence. In certain aspects, methods, systems, compositions, and kits utilize Cas12a, customized guide RNA, and an isothermal amplification buffer for polynucleotide detection. In certain aspects, methods, systems, compositions, and kits as described herein can detect SARSCoV-2, and variants thereof; hepatitis C virus (HCV), and variants thereof, human immunodeficiency virus (HIV) and variants thereof, among others. In certain aspects, methods, systems, compositions, and kits can be employed in a point-of-care setting without the use of a thermocycler.

### RELATED APPLICATION

This application claims the benefit of U.S. Provisional Application Ser. No. 63/321,408, titled “METHODS AND SYSTEMS FOR PAMLESS DETECTION OF NUCLEIC ACIDS WITH TYPE V CRISPR/CAS SYSTEMS,” filed Mar. 18, 2022. This application is incorporated herein by reference in its entirety.

### STATEMENT REGARDING FEDERALLY SPONSORED RESEARCH OR DEVELOPMENT

This invention was made with government support under Grant Numbers R21 AI156321, R21 AI168795, and R35 GM147788, awarded by the National Institutes of Health. The government has certain rights in the invention.

### SEQUENCE LISTING

The instant application contains a Sequence Listing filed in ST.26 format entitled 222111_2890_Sequence_Listing.xml created on Mar. 20, 2022. The content of the sequence listing is incorporated herein in its entirety.

### FIELD

The present disclosure relates to CRISPR/Cas complex-based systems and methods.

### BACKGROUND

The discovery of CRISPR/Cas (clustered regularly interspaced short palindromic repeats/CRISPR-associated) systems has provided new platforms and approaches to the field of genome engineering a drastically advanced applications in biology, agriculture, biotechnology, diagnostics, and treatment of genetic disorders. Originally derived from different species of bacterial adaptive immune systems, the CRISPR/Cas technology works by introducing a Cas nuclease and a short guide RNA sequence with a region complimentary to a target sequence/site that acts a guide by binding with Cas and directing the crRNA/Cas complex to a target site. This complex then acts as molecular scissors to cut the target sequence at a specific site creates double-stranded cuts in the DNA or a single-stranded cut in the RNA. This specific target recognition and cleavage is also referred to as ‘cis-cleavage’.

The development of CRISPR/Cas systems for rapid, point-of-care detection of nucleic acid targets for diagnosing diseases, such as cancer and viruses, has increased recently. The ongoing SARS-CoV-2 pandemic has vastly underscored the need for developing rapid, accurate and sensitive techniques for pathogen detection. Contemporary diagnostic methods that are based on reverse transcriptase polymerase chain reaction (RT-qPCR) are widely used, but are handicapped by their dependency on expensive reagents, sophisticated equipment, and trained personnel. CRISPR-Cas systems have emerged as a widely adopted diagnostic tool for the detection of SARS-CoV-2 and other viruses and conditions within the past year.

Class 2 type V and VI single effector Cas proteins, such as Cas12a and Cas13a, have been employed for the development of rapid, sensitive, and cost-effective detection platforms including DETECTR and SHERLOCK (Gootenberg et al., Science, 2017; Gootenberg et al., Science, 2018; Chen et al., Science, 2018; Brougton et al, Nat. Biotechnol., 2020; Young et al, NEJM., 2020) due to their robust trans-cleavage activity. The Cas12a-based DETECTR technology from Mammoth Biosciences and Cas13a-based SHERLOCK technology from Sherlock Biosciences are two CRISPR-based detection systems that are now approved by the FDA under EUA as lab-based diagnostics for detecting SARS-CoV-2 RNA. These platforms combine nucleic acid pre-amplification methods, such as RT-LAMP, RT-RPA, RT-HDA and other isothermal amplification steps, with the trans-cleavage ability of Type V and Type VI Cas effectors, for specific recognition of nucleic acid targets.

However, for the above-mentioned CRISPR-based detection methods, the pre-amplification step (such as RT-LAMP) often must be done in a separate reaction (e.g., a separate “pot”) from the CRISPR detection, since the pre-amplification has to be conducted at elevated temperatures above the melting temperature of the Cas enzyme used in the CRISPR/Cas detection step. While the optimal temperatures for RT-LAMP are around 65° C., most commercial CRISPR/Cas systems are operated at temperatures around 37° C. since many Cas enzymes become unstable at higher temperatures. The need for separate amplification and detection steps increases the time needed for the assay and can reduce sensitivity. There is a need for single pot reaction for rapid and sensitive detection of targets (e.g., diagnostics of infectious diseases, such as SARS-CoV-2).

Additionally, detection of nucleic acids such as double-stranded deoxyribonucleic acid (dsDNA) traditionally require the presence of a protospacer adjacent motif (PAM) sequence on guide nucleic acids in order for enzymatic targeting of the dsDNA sequence of interest by a CAS enzyme. This requirement for dsDNA detection is limiting, as not every sequence of interest on a dsDNA strand may be flanked or close enough to a PAM sequence to allow for efficient targeting and detection. There exists a need for systems, compositions, and methods that can utilize PAMless detection accordingly (no reliance on a PAM or anti-PAM sequence in guiding or targeting nucleic acids to bind the dsDNA strand of interest). For at least these additional reasons, there exists an additional need for improved PAMless CRISPR-based nucleic acid detection methods, systems, and compositions.

Finally, conventional methods that employ a pre-amplification step often rely on precise temperature control that must be delivered by expensive thermocyclers. Expense aside, thermocyclers are often not convenient to employ in a point-of-care situation in the field (in particular due to power and maintenance requirements). There exists an additional need for systems, kits, compositions, and methods that allow for simple CRISPR-based nucleic acid detection in the field that does not rely on a thermocycler.

### SUMMARY

Described herein are methods, systems, and kits for the PAMless detection of nucleic acids in a sample. Embodiments of the systems, methods, and kits of the present disclosure can detect target polynucleotides without the presence of a PAM sequence in the target.

Methods of the present disclosure for detecting a target polynucleotide in a sample, include the following steps: incubating the contents of a reaction vessel at a first temperature of about 60-90° C. for a first period of time; incubating the contents of the reaction vessel at a second temperature of about 25-40° C. for a second period of time; and detecting a CRISPR-generated detectable signal or detectable molecule if the target polynucleotide is present in the sample. In such methods, the reaction vessel includes: a sample comprising one or more double-stranded target polynucleotides; one or more isothermal detection components comprising an isothermal detection buffer having a pH of about 7.9 or greater; a Cas12a CRISPR-associated (Cas) enzyme; an sgRNA sequence comprising a CRISPR RNA (crRNA) sequence configured to bind to a single strand of at least one of the one or more target polynucleotides and configured to interact with the Cas12a Cas enzyme to form a CRISPR/Cas complex upon binding of the crRNA sequence to the at least one target polynucleotide; and a plurality of probes, each probe comprising an oligonucleotide element labeled with a detectable label, wherein the probe is configured to be cleaved by the Cas12a Cas enzyme when the crRNA sequence binds the target polynucleotide to generate a CRISPR-generated detectable signal or detectable molecule. According to embodiments, the target polynucleotide does not include a PAM sequence.

The present disclosure also provides one-pot nucleic acid detection systems for detecting a target polynucleotide in a sample, where the target polynucleotide does not require a PAM sequence. According to embodiments of the present disclosure, such systems can include: one or more isothermal detection components comprising an isothermal detection buffer; a Cas12a CRISPR-associated (Cas) enzyme; an sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide; and a plurality of probes, each probe comprising an oligonucleotide element labeled with a detectable label, wherein the probe is configured to be cleaved by the Cas12a Cas enzyme when the guide sequence binds the target polynucleotide to generate a detectable signal or molecule.

Embodiments of the present disclosure further include shelf-stable kits for detecting a target polynucleotide in a sample, where the target does not require a PAM sequence. Such kits can include the following components: a) an isothermal detection buffer; b) a lyophilized Cas12a CRISPR-associated (Cas) enzyme; c) a lyophilized sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide; d) a plurality of lyophilized probes, each probe comprising an oligonucleotide element labeled with a detectable label, wherein the probe is configured to be cleaved by the Cas12a Cas enzyme when the crRNA sequence binds the target polynucleotide to generate a detectable signal or molecule; and instructions for combining components a-d with a sample, incubating the sample at a first temperature for a first period of time, a second temperature for a second period of time, and detecting the detectable signal or molecule.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

The figures are further described in the description and examples below.

FIG. 1 is a schematic illustration of the effects of the PAM sequence on the trans-cleavage activity of CRISPR-Cas12a for (a) dsDNA with PAM, (b) dsDNA without PAM and (c) ssDNA without PAM.

FIG. 2 is a schematic representation of the PICNIC method for performing CRISPR-Cas12a based detection of non-canonical PAM containing DNA using high heat and pH to denature the dsDNA into separate single-strands.

FIGS. 3A-3B illustrate the trans-cleavage activity (FIG. 3A) of 3 different Cas12a orthologs (Lb, As, Er) at pH=12 and the isoelectric points (FIG. 3B) of several different Case12a orthologs. Of the 3 tested, LbCas12a has the highest isoelectric point and shows highest activity at pH=12.

FIG. 4 illustrates fold change at t=30 min using a PAM library of dsDNA activators containing different PAM sequences (changing 1^{st} through 3^{rd} position of the PAM sequence) comparing wild type CRISPR detection vs. PICNIC methods and systems of the present disclosure. The figures show that PICNIC is able to detect non-canonical PAM sequences.

FIG. 5 illustrates fold change at t=30 min using a PAM library of dsDNA activators containing different PAM sequences changing the 4^{th} position of the PAM sequence comparing wild type CRISPR detection vs. PICNIC methods and systems of the present disclosure and showing that PICNIC is able to detect non-canonical PAM sequences.

FIG. 6 is a schematic illustrating approach for testing PICNIC methods and systems of the present disclosure. A dsDNA activator is designed to contain fluorophore on one strand and quencher on the complementary strand. If the DNA is in double-stranded form, the fluorophore is quenched, and no fluorescence is observed. The illustration shows that at higher temperature and/or in PICNIC buffer at higher pH even after cooled to room temperature, the dsDNA denatures into single-strands and emits fluorescence.

FIGS. 7A-7B illustrate effect of temperature and pH buffer (pH 12) on detection of dsDNA with methods and systems of the present disclosure. FIG. 7A (top) illustrates a fluorophores-quencher tagged dsDNA where fluorescence is quenched when strands are annealed, and fluorescence increases with increasing percent of ssDNA. The image in FIG. 7A (bottom) and graph in FIG. 7B (n=3) show the effect of temperature on double-stranded DNA in PICNIC buffer (pH=12) vs water (pH˜7) (n=3). In PICNIC buffer, the dsDNA denatures into single-strands and emits fluorescence at temperatures above about 55° C.

FIGS. 8A-8B illustrate effect of pH on detection of dsDNA with methods and systems of the present disclosure. FIG. 8A (top) illustrates a fluorophores-quencher tagged dsDNA where fluorescence is quenched when strands are annealed, and fluorescence increases with increasing percent of ssDNA. The image in FIG. 8A (bottom) and graph in FIG. 8B (n=3) show that fluorescence increased with increasing pH buffer vs. water, indicating increased amount of ssDNA with increased pH.

FIG. 9 illustrates application of embodiments of the methods and systems of the present disclosure for detection of diverse hepatitis C virus (HCV) genotypes. Sequence alignment of HCV-1a (top) and HCV-1b shows sites within the genome having numerous mutations (shaded boxes) that can be targeted to discriminate between the two variants, but where no canonical ‘TTV’ PAM sequence is available to perform classic CRISPR-based detection, making it a good candidate for detection with the PICNIC assays of the present disclosure.

FIGS. 10A-10C illustrate detection of hepatitis C virus (HCV) variants with embodiments of systems and methods of the present disclosure, called PICNIC. FIG. 10A is a schematic illustrating and embodiment of elements and steps of the systems and methods. FIGS. 10B and 10C are graphs showing detection of HCV variants 1a and 1b with PICNIC as compared to standard CRISPR-based detection methods.

FIGS. 11A-11D illustrate optimization of embodiments of PICNIC assay for different pH values (FIG. 11A), various buffers/additives (FIG. 11B), incubation times (FIG. 11C), and incubation temperatures (FIG. 11D).

FIG. 12 is a bar graph illustrating comparison of detection of PAMless dsDNA with CRISPR-ENHANCE vs PICNIC (embodiments of systems and methods of the present disclosure) and showing superior detection with PICNIC assay. Plot of Fold change of the fluorescence intensity w.r.t. the NTC is shown.

FIGS. 13A-13B illustrate detection of non-PAM containing HIV drug-resistant mutants with methods and systems of the present disclosure. FIG. 13A shows an alignment of the nucleotide and peptide sequence of wildtype HIV-K103 (top) and a single point mutation variant HIV-N103 (bottom) of HIV illustrating the location of the point mutation without a nearby canonical pam site. FIG. 13B illustrates detection and distinction of both variants with an embodiment of the PICNIC assay of the present disclosure compared to with standard Cas12a assay.

The figures are also further discussed in the description and examples below.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Prior to describing the various embodiments, the following definitions are provided and should be used unless otherwise indicated.

### Definitions

In describing and claiming the disclosed subject matter, the following terminology will be used in accordance with the definitions set forth below.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA).

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the terms “guide polynucleotide,” “guide sequence,” or “guide RNA” (gRNA or sgRNA) as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide (gRNA or sgRNA) can include a nucleotide sequence that is complementary to a target DNA sequence. This portion of the guide sequence can be referred to as the complementary region of the guide RNA or the CRISPR RNA (crRNA). Another portion of the guide sequence serves as a binding scaffold for the CRISPR-associated (Cas) nuclease. For some Cas enzymes, this portion of the guide sequence can be referred to as the tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451).

A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, the terms “optional” or “optionally” means that the subsequently described event or circumstance can or cannot occur, and that the description includes instances where said event or circumstance occurs and instances where it does not.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a K_{d} of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. In some embodiments of the present disclosure viral vectors are used, such as single stranded viral vectors, such as adeno-associated virus (AAV) vectors.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “transforming” when used in the context of engineering or modifying a cell, refers to the introduction by any suitable technique and/or the transient or stable incorporation and/or expression of an exogenous gene in a cell.

As used herein, “selectable marker” refers to a gene whose expression allows one to identify cells that have been transformed or transfected with a vector containing the marker gene. For instance, a recombinant nucleic acid may include a selectable marker operatively linked to a gene or insert of interest and a promoter, such that expression of the selectable marker indicates the successful transformation of the cell with the gene or insert of interest. Examples of selectable markers include, but are not limited to, DNA and/or RNA segments that contain restriction enzyme sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; epitope tags (e.g. FLAG- and His-tags), and, the inclusion of a DNA sequences required for a specific modification (e.g., methylation) that allows its identification. Other suitable markers will be appreciated by those of skill in the art.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

### DISCUSSION

In accordance with the purpose(s) of the present disclosure, as embodied and broadly described herein, embodiments of the present disclosure, in some aspects, relate to systems and methods of detecting a target polynucleotide without the use of a PAM sequence. Systems, methods, kits, and assays of the present disclosure are sometimes referred to herein by the term PICNIC (PAMless Identification of Nucleic Acids with CRISPR/Cas). In some aspects, embodiments of the present disclosure relate to systems, methods, compositions, and kits that provide for PAMless isothermal detection of nucleic acids (in particular dsDNA and single-stranded (ssDNA) from samples of interest). In certain aspects, embodiments of the present disclosure relate to systems, methods, compositions, and kits that provide for PAMless isothermal detection of nucleic acids (in particular dsDNA and single-stranded (ssDNA)) from a sample in a single-pot utilizing CRISPR technology (in particular Cas12a).

According to some embodiments of the present disclosure, systems and methods of the present disclosure may combine isothermal amplification and PAMless CRISPR/Cas detection in a single pot at elevated temperatures for rapid and accurate detection. The present disclosure also includes kits for use in the methods of the present disclosure. According to some aspects, the present disclosure provides methods, systems, and kits for rapid and accurate detection of infectious agents, including, but not limited to viruses such as SARS-CoV-2, hepatitis C virus (HCV), variants of HIV, and other diseases and conditions. In some embodiments, the methods, systems, and kits of the present disclosure are able to detect/distinguish variants of SARS-CoV-2, HCV, and/or HIV. Additional aspects of the current disclosure are provided in the discussion below.

### Overview

The breakthrough of CRISPR/Cas (clustered regularly interspaced short palindromic repeats/CRISPR-associated) systems has transformed the slow-progressing field of genome engineering with diverse applications in biology, agriculture, biotechnology, diagnostics, and treatment of genetic disorders. Originally derived from adaptive immune systems of different species of bacteria, the initial iterations of CRISPR/Cas9 technology enabled genetic engineering by providing targeted endonuclease activity at sites determined by a combination of the PAM (protospacer adjacent motif) requirement of each specific Cas enzyme complexed with a sgRNA (single guide RNA) composed of a variable length crRNA sequence complementary to the target site fused to a Cas specific sequence, and for some Cas enzymes, a tracrRNA sequence. For Cas9 enzymes, this complex then creates double-stranded cuts in the DNA or a single-stranded cut in the RNA. This specific target recognition and cleavage is also referred to as “cis-cleavage”.

Some CRISPR/Cas systems also exhibit collateral non-specific cleavage or “trans-cleavage” of single-stranded nucleic acid immediately after the specific target recognition or ‘cis-cleavage’ of the target. This trans cleavage activity can be harnessed for diagnostic applications. CRISPR is currently classified into two classes based on whether they require multiple Cas effector proteins (Class 1) or a single Cas effector (Class 2). Class 1 includes systems that require multiple Cas effector proteins. Class 2 systems are of high interest as they are based on single Cas effector proteins and crRNA and are further divided into type II, V, and VI. Type II includes, Cas9, which has been studied for gene editing applications. The type V and VI CRISPR/Cas systems such as CRISPR/Cas12, CRISPR/Cas13, and CRISPR/Cas14 (abbreviated as CRISPR/Cas12-14) are emerging as powerful tools for nucleic acid detection and applications in gene and RNA editing.

Both type V and VI systems are of special interest as they are based on single Cas effector proteins and can cleave dsDNA and ssRNA, some (such as Cas12a) without requiring a longer tracrRNA, and they possess a trans-cleavage activity that can be applied for diagnostics. Using single-stranded nucleic acid-based FRET reporters, the trans-cleavage activity of type V and VI systems has been applied for the detection of target dsDNA and ssRNA targets at low nM (1-10 nM) concentrations without further amplification. However, no trans-cleavage signal could be observed below 10 nM of dsDNA without any DNA amplification using the Acidaminococcus sp. derived AsCas12a or below 1 nM (100 fmols) using Lachnospiraceae bacterium derived LbCas12a. However, by coupling these detection systems with isothermal amplification, low aM concentration of cell-free tumor DNA in lung cancer and viral RNA in human saliva and blood samples have been achieved (Tsou J H, Leng Q, Jiang F. A CRISPR Test for Rapidly and Sensitively Detecting Circulating EGFR Mutations. Diagnostics (Basel). 2020; 10(2):114. Published 2020 Feb. 19. doi:10.3390/diagnostics10020114).

Furthermore, previous methodologies have relied on the presence of PAM (or anti-PAM) sequences in the target strand for guiding nucleic acids to enable detection of regions of interest on nucleic acids from a sample. The requirement of the inclusion of PAM or anti-PAM sequences limits the applications of the technology from a primer/guide nucleic acid perspective and a target perspective. While some enzyme engineering has been performed on Cas12a enzymes, such as LbCas12a (Tóth E, Varga É, Kulcsár P I, et al. Improved LbCas12a variants with altered PAM specificities further broaden the genome targeting range of Cas12a nucleases. Nucleic Acids Res. 2020; 48(7):3722-3733. doi:10.1093/nar/gkaa110), to relax the requisite PAM requirement, there still exists a desire for simple PAMless systems for detection of targets without a nearby PAM sequence.

As described herein, systems, compositions, kits, and methods employing Cas12a enzymes (including, but not limited to, LbCas12a, AsCas12a, and ErCas12a) allow for simple PAMless detection of nucleic acids, in particular the isothermal detection of DNA from a sample (which can be done in a. single pot, and in certain aspects, multiplexed with isothermal amplification, if appropriate).

As described in greater detail in the examples below, the methods and systems of the present disclosure combine the use of Cas12a enzymes (such as, but not limited to, LbCas12a, AsCas12a, and ErCas12a) for detection of nucleic acid targets in a sample at higher temperature with or without an isothermal amplification step (e.g., LAMP, RT-LAMP) for detection of polynucleotide targets (including, but not limited to, RNA viruses such as SARS-CoV-2, HIV, and HCV; other viruses such as Bluetongue Virus (BTV), spread by Culicoides; and pathologies of parasitic origin, for example malaria, which is spread by organisms of the Plasmodium group) in a single pot reaction.

Specificity of enzymes for a target can also be enhanced at higher temperatures, and some Cas12a variants have been identified that can function at different temperatures (see, for instance, Nguyen, Long T., et al., A Combinatorial Approach towards Adaptability of 22 Functional Cas12a Orthologs for Nucleic Acid Detection in Clinical Samples; 2021, https://www.medrxiv.org/content/I0.1101/2021.07.21.21260653v1, which is hereby incorporated by reference herein). Therefore, in some embodiments LbCas12b (or other Cas12a enzymes as described herein) can also be combined and/or multiplexed with other Cas enzymes (such as Cas12a) to detect two different targets at different temperatures. For example, the activity of Cas12b can be blocked by incorporating an inhibitor (e.g., an anti-CRISPR or blocked sgRNAs). The inhibitor can be configured to be removed or degraded at higher temperatures, such as above 60° C., above 62° C., or at or above 65° C., thereby activating the BrCas12b enzyme for detection of a target.

The LbCas12b enzyme, like various Cas12a enzymes, appears to be compatible with other CRISPR/Cas technologies. This includes CRISPR Chain Reaction technologies, where two or more CRISPR/Cas complexes are combined in a chain reaction, where one complex is activated and can activate a second inactive CRISPR/Cas complex. Such systems are described in greater detail in PCT/US2021/034971, which is hereby incorporated by reference herein. In some embodiments, a LbCas12b enzyme can be employed in such systems and methods, such as combined with itself or other Cas12a enzymes (combined or substituted with any one or more of other Cas12a enzymes, such as, but not limited to ArCas12a, AsCas12a, BfCas12a, BoCas12a, BsCas12a, CMaCas12a, CmtCas12a, ErCas12a, FnCas12a, HkCas12a, Lb2Cas12a, Lb5Cas12a, MbCas12a, Mb2Cas12a, Mb3Cas12a, MiCas12a, Pb2Cas12a, PcCas12a, PdCas12a, PrCas12a, PxCas12a, TsCas12a, other Cas12b enzymes (such as, but not limited to, AapCas12b, AacCas12b, BhCas12b, AkCas12b, EbCas12b, LsCas12b, BthCas12b, BvCas12b, AaCas12b, and the like), Cas12a enzymes, and/or Cas13b enzymes.

Such combined systems can create an exponential amplification chain reaction with increased specificity and sensitivity, including elevated temperature detection.

In yet other embodiments, the LbCas12b enzyme can also be combined in a multiplexed fashion with different Cas enzymes based on sequence cleavage preferences, multiplexing based on temperature, or combined with aptamers for detecting small molecules. All such systems can be incorporated with a variety of reporter systems based on one or more of fluorescence, luminescence, color change, product formation redox reaction, pH change, surface reaction or cleavage, change in electrical conductivity, resistance, and/or impedance.

As described in greater detail in the Examples and figures below, embodiments and variations of the one-pot CRISPR/Cas detection systems, methods, and kits include various elements that can be combined in a single-pot for quick and accurate detection of a target polynucleotide in a sample. Some elements of the methods, systems, and kits are described below.

### One-Pot Cas12a CRISPR/Cas Nucleic Acid Detection Systems and Methods:

In embodiments, the one-pot nucleic acid detection systems of the present disclosure include one-pot nucleic acid detection systems for detecting a target polynucleotide (for example dsDNA or ssDNA) in a sample. In such aspects, a Cas enzyme, such as a Cas12a enzyme (such as, but not limited to, LbCas12a, AsCas12a, or ErCas12a) can be employed to detect a nucleic acid from a sample using elevated temperatures for a time in combination with isothermal detection components, such as an isothermal detection buffer, such as described below (e.g., a high-salt and/r high-pH buffer (for example, in embodiments, the buffer is ThermoPol® reaction buffer from New England Biolabs® (NEB).). In some embodiments, the buffer can be high-pH water (e.g., basic water at a pH of about 8.8 or higher).

In certain aspects, systems and methods of the present disclosure may also be combined with isothermal amplification components and target nucleic acid amplification protocols. The systems and methods of the present disclosure can combine isothermal amplification approaches and CRISPR/Cas detection to detect a target polynucleotide (including, for instance, a target polynucleotide lacking a PAM sequence) in a one-pot reaction. The isothermal amplification elements of the methods and systems can include a set of isothermal amplification components including isothermal amplification enzymes (e.g., polymerases, reverse transcriptase, etc., as needed) and isothermal amplification primers configured to recognize and amplify the target polynucleotide. The CRISPR/Cas detection portion of the systems and methods include a thermostable Cas enzyme, such as a Cas12a enzyme (for example, LbCas12a, AsCas12a, or ErCas12a), a single guide RNA (sgRNA) sequence having a CRISPR RNA (crRNA) sequence configured to bind to the target polynucleotide of interest, and probes configured to be cleaved by the Cas12a enzyme and generate a detectable signal or molecule when the crRNA sequence binds the target polynucleotide in the sample. Some of these elements and additional elements will be described in great detail below as well as in the examples and figures. The present disclosure also provides kits that include shelf stable components of the systems of the present disclosure that can be used in the methods of the present disclosure, as described in greater detail below.

### Isothermal Amplification/Detection Platforms and Components

In embodiments, methods, systems and kits of the present disclosure can include one or more isothermal detection components and isothermal amplification components, including, but not limited to isothermal detection buffers and/or isothermal amplification primers and isothermal amplification enzymes.

The systems of the present disclosure can include one or more isothermal detection component(s), such as an isothermal detection buffer. In embodiments the isothermal detection buffer includes a buffer having a high-pH (e.g., a basic pH of about 8 or higher at room temperature, or before heating). In embodiments the isothermal detection buffer can have overall salt concentration of about 10 mM to about 500 mM salt. In embodiments, the isothermal detection buffer is a high-salt, high-pH buffer. In embodiments the isothermal amplification/detection buffer can be, but is not limited to selected from, but not limited to Tris-HCl, (NH₄)₂SO₄, KCl, MgSO₄ Triton® X-100, ThermoPol® from New England Biolabs®, or water with a pH of about 8 or higher. In embodiments, the systems of the present disclosure also include isothermal amplification components (described below). In embodiments, the isothermal amplification components and isothermal detection components are compatible and amplification and detection can occur in one pot.

Isothermal amplification platforms are platforms that allow for amplification of a target polynucleotide in a sample without the high-temperature thermocycling and expensive equipment required for PCR amplification. Isothermal amplification is performed at moderately high temperatures of about 60-70° C. (typically about 60-65° C.), which can be maintained in relatively simple and readily available equipment, including a warm water bath. The reaction components for isothermal amplification include sets of isothermal amplification primers and thermostable enzymes, such as polymerases, and, in the case of RNA amplification, reverse transcriptase for converting RNA targets to DNA for amplification. The isothermal amplification primers include sets of primers for recognizing, binding, an initiating amplification of the target polynucleotide.

Common platforms for isothermal amplification include loop-mediated isothermal amplification (LAMP) and reverse-transcription LAMP (RT-LAMP). LAMP is typically used for amplification/detection of DNA and includes thermostable DNA polymerases, where RT-LAMP is used for amplification/detection of RNA and includes both thermostable reverse transcriptase and thermostable DNA polymerase. Both LAMP and RT-LAMP include sets of LAMP primers. LAMP primers typically include a set of 4 to 6 thermostable primers configured to bind to different small target sequences of the target polynucleotide to initiate amplification. In embodiments the LAMP primers include at least a forward internal primer (FIP), a backward internal primer (BIP), a forward outer primer (sometimes referred to as F3), and a backward outer primer (sometimes referred to as B3). In embodiments, the LAMP primers can also include a set of loop primers.

LAMP primers for specific target polynucleotides can be designed using software programs. In embodiments, the target polynucleotide can be a DNA or RNA associated with a specific condition/disease, such as cancer, genetic disease, or infectious agent (e.g., bacteria, virus, fungal, etc.). In embodiments, the target polynucleotide is a virus, such as SARS-CoV-2, HIV, HCV, Chagas, malaria, bluetongue virus, and the like. LAMP primers can be designed for recognition and amplification of any such DNA or RNA targets. Additional description of isothermal amplification enzymes and primers, such as LAMP/RT-LAMP primers and enzymes, is provided in the examples below.

In embodiments, the isothermal amplification components in the reaction vessel also include an isothermal amplification buffer compatible with the isothermal detection components, any other isothermal amplification components and the Cas12a Cas enzyme. In embodiments, the buffer is a buffer from New England Biolabs® (NEB). In embodiments it is NEBuffer 2.1 or WarmStart® Multi-Purpose LAMP/RT-LAMP 2× Master Mix (with UDG) (“NEB LAMP Master Mix”) or TwistAmp Basic Kit (From Twist). In embodiments, the buffer is ThermoPol® reaction buffer from New England Biolabs® (NEB).

In some embodiments of the present disclosure, the set of isothermal amplification components also includes an isothermal amplification reporter that produces a detectable signal upon amplification of the target sequence, such that the signal becomes detectable after sufficient amplification of the target sequence, indicating its presence in the sample. The isothermal amplification reporter is configured to produce an amplification-generated detectable signal or detectable molecule upon amplification of the target polynucleotide. In embodiments, the amplification-generated detectable signal or molecule of the isothermal amplification reporter is different and distinguishable from the CRISPR-generated detectable signal or detectable molecule produced by cleavage of the probes (described in greater detail below). In embodiments, the isothermal amplification reporter is an amplification-generated dye or other detectable signal. In embodiments, the isothermal amplification reporter is a SYTO dye, including but not limited to SYTO9, SYTO62, SYTO17, SYTO59, and SYTO60-64.

### CRISPR-Associated Enzyme

CRISPR-associated (Cas) enzymes (also known as CRISPR effector protein) are enzymes that can bind to a guide RNA (sgRNA) and to a complementary target polynucleotide sequence, forming a CRISPR/Cas complex, and can cleave the target sequence (cis cleavage). The Cas enzymes of the present disclosure possess both cis- and trans-cleavage activity, where trans-cleavage activity is activated upon binding of the CRISPR/Cas complex with the target sequence. Activation of the trans cleavage activity allows cleavage of probes also included in the reaction mixture, such that the probes produce a detectable signal or molecule that indicates the presence of the target sequence.

In embodiments of the present disclosure, the Cas enzymes are Cas12a enzymes. In some embodiments, the Cas12a enzymes are from a digestive-tract bacterium Lachnospiraceae. The Lachnospiraceae Cas enzyme, LbCas12a exhibits outstanding stability at high temperatures (up to about 50-55° C. or slightly greater) and, as described in the Examples below, was found to exhibit robust trans cleavage activity at these elevated temperatures, which is suitable for coupling with an isothermal detection reaction according to the present disclosure and/or isothermal amplification reaction, such as LAMP and/or RT-LAMP.

In embodiments, the LbCas12a of the systems and methods of the present disclosure is derived from subspecies Lachnospiraceae sp. (for example, GenBank ID: MW477886.1). In embodiments, the LbCas12a enzyme has trans cleavage activity at temperatures up to about or higher than about 55° C., with robust activity between about 37 and 55° C.) and a melting temperature of about 50° C.

In embodiments of the present disclosure, the Cas enzymes (Cas12a in particular) are from a digestive-tract bacterium Acidaminococcus. The Acidaminococcus Cas enzyme, AsCas12a exhibits outstanding stability at high temperatures (up to about 70° C. or greater) and, as described in the Examples below, was found to exhibit robust trans cleavage activity at these elevated temperatures, which is suitable for coupling with an isothermal detection reaction according to the present disclosure and/or isothermal amplification reaction, such as LAMP and/or RT-LAMP.

In embodiments, the AsCas12a of the systems and methods of the present disclosure is derived from subspecies Acidaminococcus sp. (for example, GenBank ID: MW477885.1). In embodiments, the AsCas12a enzyme has trans cleavage activity at temperatures up to about or higher than about 55° C., with robust activity between about 37 and 55° C.) and a melting temperature of about 50° C.

In embodiments of the present disclosure, the Cas enzymes (Cas12a in particular) are from a digestive-tract bacterium Eubacterium rectale. The Eubacterium rectale Cas enzyme, ErCas12a exhibits outstanding stability at high temperatures (up to about 70° C. or greater) and, as described in the Examples below, was found to exhibit robust trans cleavage activity at these elevated temperatures, which is suitable for coupling with an isothermal detection reaction according to the present disclosure and/or isothermal amplification reaction, such as LAMP and/or RT-LAMP.

In embodiments, the ErCas12a of the systems and methods of the present disclosure is derived from subspecies Eubacterium rectale (for example, GenBank ID: MH347339.1). In embodiments, the ErCas12a enzyme has trans cleavage activity at temperatures up to about or higher than about 55° C., with robust activity between about 37° C. and 55° C.) and a melting temperature of about 48° C. It is note that temperature parameters for ErCas12a may be about the same as other Cas12a enzymes.

sgRNA and crRNA

The guide polynucleotide, also called guide RNA or sgRNA, of the present disclosure includes both a guide CRISPR RNA (crRNA) sequence and a conserved sequence. The crRNA sequence is configured to bind to a target polynucleotide, and the conserved sequence (and tracrRNA sequence for some Cas enzymes, e.g., cAs12a, which does not need a tracrRNA sequence) is conserved among sgRNA from closely related bacterial species and is configured to act as a scaffold for complexing with the Cas12a Cas enzyme (for example LbCas12a, AsCas12a, and ErCas12a) upon binding of a target sequence to form a CRISPR/Cas complex.

In some embodiments, a sgRNA (crRNA with or without tracrRNA sequence, where needed) is about 130 base pairs. In embodiments, the length of the guide sequence of the crRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide sequence is less than about 75, 50, 45, 40, 35, 30, 25, 20, 15, 12, or fewer nucleotides in length. In one embodiment, the guide sequence is 10-30 nucleotides long.

In embodiments, the target polynucleotide can be a DNA (for example dsDNA or ssDNA) or RNA associated with a specific condition/disease, such as cancer, genetic disease, or infectious agent (e.g., bacteria, virus, fungal, etc.). In embodiments, the target polynucleotide is from a virus, such as, but not limited to SARS-CoV-2, HIV, HCV, Chagas, malaria, bluetongue virus, and the like. In embodiments the target polynucleotide is a PAMless double stranded DNA (dsDNA) target (e.g., a segment of dsDNA where there is not a canonical PAM sequence on the non-target strand for recognition by standard Cas enzymes). In embodiments the crRNA can be complimentary to and configured to bind a complimentary region on the target DNA or RNA of interest to detect the specific condition/disease, such as cancer, genetic disease, infection agent (e.g., bacteria, virus, etc.) and the like.

In embodiments, the crRNA binds the amplified target polynucleotides and forms a CRISPR/Cas complex with the thermostable CAS12a to activate the trans cleavage activity of the Cas12, which cleaves probes in the reaction vessel to create a detectable signal and indicate the presence of the target polynucleotide in the sample, thereby providing information that can be utilized for the diagnosis of the specific condition/disease associated with the target polynucleotide.

In embodiments, the target polynucleotide in the sample can be amplified by the isothermal amplification components, and then the CrRNA binds the amplified target polynucleotides and forms a CRISPR/Cas complex with the thermostable CAS12a to activate the trans cleavage activity of the Cas12, which cleaves probes in the reaction vessel to create a detectable signal and indicate the presence of the target polynucleotide in the sample, thereby diagnosing the specific condition/disease associated with the target polynucleotide.

In embodiments the target polynucleotide is from a virus such as, but not limited to, SARS-CoV-2, HIV, HCV, Chagas, malaria, bluetongue virus, etc., and the CRISPR/LbCas12a complex binds the target polynucleotide, activating trans cleavage activity, cleaving the probes, and detecting the presence of the virus in the sample.

In embodiments, the guide polynucleotide can also be designed according to aspects of other technologies, such as CRISPR-ENHANCE described in PCT application PCT/US2020/059577 (publication WO 2021/092519), and “CRISPR CHAIN REACTION SYSTEMS AND METHODS FOR AMPLIFYING THE DETECTION SENSITIVITY OF CRISPR-BASED TARGET DETECTION” described in PCT application PCT/US2021/034971 filed on May 28, 2021, both of which are incorporated by reference as fully set forth herein. Additional technologies that can be utilized/paired with the methods/systems of the present disclosure include the CRISPR based technologies described in, but not limited to, the following applications, each entry of which are incorporated herein by reference as if fully set forth herein: PCT/US2022/079420; PCT/US2022/076926; U.S. patent application Ser. No. 17/928,400; and U.S. patent application Ser. No. 17/775,206.

### Probe

As used herein, a “probe” refers to a polynucleotide-based molecule that can be cleaved by an activated CRISPR-associated (Cas) enzyme with a trans-cleavage activity to produce a detectable signal or a detectable molecule. A detectable signal may be any signal that can be detected using optical, fluorescent, chemiluminescent, electrochemical or other detection methods known in the art. The probe comprises an oligonucleotide element. In one embodiment, a first end of the oligonucleotide element in the probe is linked to a fluorophore; and a second end of the oligonucleotide element in the probe is linked to a quencher of the fluorophore. In one embodiment, the probe further comprises biotin. In some embodiments of the present disclosure, the probes are configured to be cleaved by a Cas enzyme, such as Cas12a enzyme (for example LbCas12a, AsCas12a, or ErCas12a) in crRNA/Cas complex, such that the detectable signal or molecule can be produced upon binding of the crRNA/CAs12a complex to the target polynucleotide.

Quenching of the fluorophore can occur as a result of the formation of a non-fluorescent complex between the fluorophore and another fluorophore or nonfluorescent molecule. This mechanism is known as ground state complex formation, static quenching, or contact quenching. Accordingly, the oligonucleotide element may be designed so that the fluorophore and quencher are in sufficient proximity for contact quenching to occur. Fluorophores and their cognate quenchers are known in the art and can be selected for this purpose by one having ordinary skill in the art. Upon activation of the Cas12a enzyme disclosed herein, the oligonucleotide-based probe is cleaved thereby severing the proximity between the fluorophore and quencher needed to maintain the contact quenching effect. Accordingly, detection of the fluorophore may be used to determine the presence of a target molecule in a sample. In one embodiment, the fluorophore is selected from the group consisting of FITC, HEX and FAM, and the quencher is selected from the group consisting of BHQ1, BHQ2, MGBNFQ, and 3IABkFQ. In one embodiment, a first end of the oligonucleotide element in the probe is linked to a fluorophore; a second end of the oligonucleotide element in the probe is linked to a quencher; and the probe further comprises biotin. In one embodiment, a fluorophore-quencher probe is within the crRNA and the quencher was only cleaved in the presence of a target polynucleotide.

A detectable molecule may be any molecule that can be detected by methods known in the art. In one embodiment, the detectable molecule is one member of a binding pair and can be detected by binding to another member of the binding pair. Examples of binding pairs include, but are not limited to, antibody-antigen pairs, enzyme-substrate pairs, receptor-ligand pairs, and streptavidin-biotin. In one embodiment, the probe is a HEX-FQ) reporter.

In one embodiment, the oligonucleotide element in the probe is ssDNA, since Cas12 trans cleavage can cut DNA. Since Cas12 enzymes preferentially cleaves DNA with an A/T rich sequence, in embodiments the oligonucleotide element of the probe includes at least 55%, 60%, 65%, 70%, 75%, 80%, 90%, or 95% of A and/or T. In embodiments the oligonucleotide element of the probe is a ssDNA and is about 80% of A and/or T. In some embodiments the oligonucleotide element is TA-rich or TA-only, and is about 2-10 nucleotides. In one embodiment, the ssDNA consists of A and/or T. In one embodiment, the oligonucleotide element is TTATT. In some embodiments, the oligonucleotide element is primarily or only T “poly T”, and in some embodiments it is polyT and about 2-10 nucleotides in length. In embodiments it is an “8-mer poly T” (TTTTTTTT).

In one embodiment, the probe can be, but is not limited to a HEX-FQ reporter, such as HEX-TTATT-FQ. In another embodiment, the probe comprises HEX-polyT-Quencher (HEX-FQ). In embodiments, the probe can also be and FITC probe or a Cy5 probe, such as, but not limited to FITC-polyT-Quencher and Cy5-PolyT-quencher.

### Methods

The present disclosure also includes methods of detecting a target polynucleotide in a sample. In embodiments, the present disclosure includes methods for PAMless detection of target polynucleotides in a sample. In some embodiments, the methods are carried out in a single “pot” (e.g., reaction vessel).

In embodiments of methods as described herein, the sample can include one or more target nucleotides. In embodiments of methods as described herein, the sample can be a biological sample that can be lysed according to first temperatures as disclosed below (for example a biological sample containing one or more living cells having a polynucleotide of interest (target polynucleotide) within one or more lipid bilayers of the living cell). In embodiments of methods according to the present disclosure, the sample can be a saliva, sputum, blood, urine, or other sample from a human. In embodiments of methods according to the present disclosure, the sample can contain a virus, bacterium, parasite or other microorganism, containing one or more target polynucleotides (e.g., polynucleotides of interest). In embodiments of methods as described herein, the sample can be a mammalian sample (for example from a human, or livestock or wild mammals, such as sheep, cattle, deer, and the like).

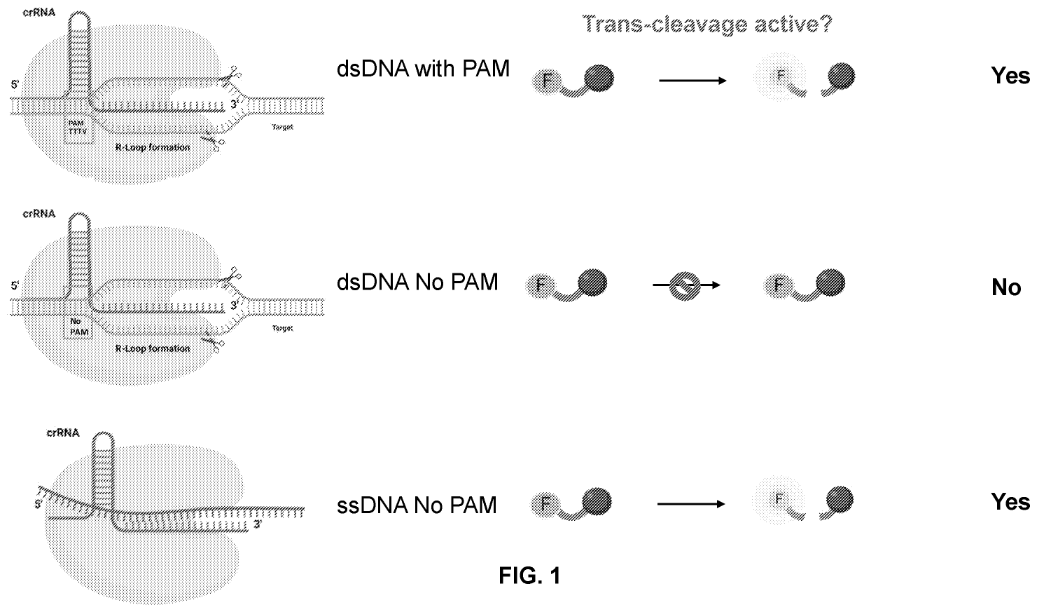

In embodiments, the target nucleotide of interest in the sample can be a double or single stranded nucleotide. In embodiments, the target of interest is a double stranded DNA that lacks a canonical PAM sequence (e.g., a protospacer adjacent motif sequence located adjacent to the crRNA binding site on the non-binding strand of the target sequence. As illustrated in FIG. 1, for traditional CRISPR/Cas detection, the PAM motif is needed for the detection of double-stranded DNA with standard CRISPR-Cas12a systems. In such systems the trans-cleavage activity of Cas12a is turned on in the presence of a dsDNA target sequence containing the PAM recognition site (FIG. 1, top), but is OFF when PAM is absent (FIG. 1, middle). Interestingly, single stranded DNA targets can be detected even in the absence of a PAM site (FIG. 1, bottom). However, for detection of targets that are present in a sample as dsDNA in such a manner would necessitate keeping the strands separated. While the strands can be separated at high temperatures, keeping the strands separated typically requires temperatures higher than the melting temperatures of most Cas enzymes. Cas enzymes that work at higher temperatures are known, as described herein; however, other Cas12a enzymes that work best a lower temperatures are more convenient, readily available, and cost effective. Thus, there is a need for methods and systems that will work for detection of PAMless double stranded targets without the need for high-temperature resistant Cas enzymes.

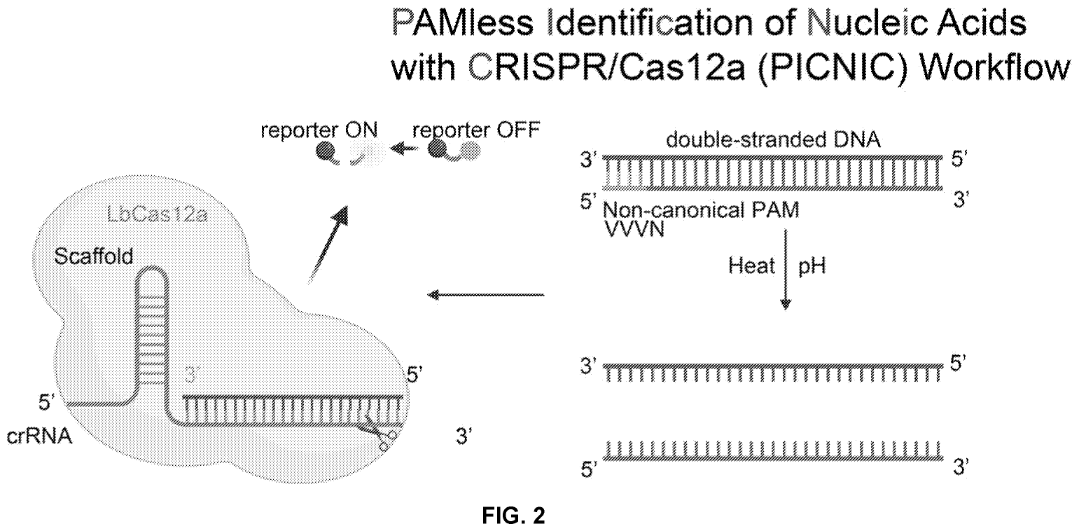

FIG. 2 illustrates embodiments of methods and systems of the present disclosure for PAMless Identification of Nucleic Acids with CRISPR/Cas, also referred to herein as PICNIC. As illustrated, PAM-less dsDNA (also referred to as sequences with non-canonical PAM), are subject to elevated temperatures and pH to separate the strands, which then allows detection of the single stranded DNA by binding of crRNA and the Cas enzyme to the non-PAM containing single strand. For reference, the typical or canonical PAM sequences that are used for recognition by wild type CRISPR/Cas12a systems are typically T and C containing PAM sequences, not A/G containing sequences. The PICNIC methods and systems of the present disclosure allow detection of the dsDNA targets even without such T/C containing canonical PAM sequences typically required for detection with CRISPR/Cas12a systems.

According to embodiments of methods of the present disclosure, a sample can first be heated to a first temperature (e.g., about 60° C. or higher, such as described below) in the presence of isothermal detection components, including, but not limited to an isothermal detection buffer, such as, but not limited to a high-salt buffer, a high-pH (e.g., basic, having a pH of about 8 or more) buffer or both (or a buffer that is both high salt and high pH). In embodiments the isothermal detection buffer can have overall salt concentration of about 10 mM to about 500 mM salt. The sample can then be cooled to a second temperature and one or more target nucleotides detected with a Cas12a enzyme. In embodiments, the second temperature is substantially cooler than the first temperature (e.g., by at least 25-30 degrees C. or more).

In embodiments of methods of the present disclosure, the first temperature is about 60° C. to about 95° C.; about 65° C. to about 95° C.; about 65° C. to about 90° C.; about 65° C. to about 85° C.; about 65° C.; about 65° C. to about 80° C.; about 65° C. to about 75° C.; about 70° C.; about 70° C. to about 95° C.; about 75° C. to about 95° C.; about 80° C. to about 95° C.; about 85° C. to about 95° C.; or about 90° C. to about 95° C. In embodiments, the first temperature is about 85° C. In embodiments, the first temperature is applied for an incubation period of about 2 minutes to about 30 minutes.

In embodiments, the isothermal detection buffer is a high-pH buffer having an initial pH (before heating to the first temperature, second temperature, or both) of about 8 to about 14; about 8.5 to about 14; about 9 to about 14; about 9.5 to about 14; about 10 to about 14; about 10.5 to about 14; about 11 to about 14; about 11.5 to about 14; about 12 to about 14; about 12.5 to about 14; about 13 to about 14; about 13.5 to about 14; about 8 to about 13.5; about 8 to about 13; about 8 to about 12.5; about 8 to about 12; about 8 to about 11.5; about 8 to about 11; about 8 to about 10.5; about 8 to about 10; about 8 to about 9.5; about 8 to about 9; about 8 to about 8.8; about 8.5; or about 8.3 to about 8.8.

In embodiments according to methods of the present disclosure, the isothermal detection component(s) can include a buffer, also referred to herein as an isothermal detection buffer. In embodiments, the isothermal detection buffer can be selected from, but not limited to Tris-HCl, (NH₄)₂SO₄, KCl, MgSO₄ Triton® X-100, ThermoPol® from New England Biolabs®, or water with a pH of about 8 or higher. In embodiments, the isothermal detection components can be selected from: about 0.2 mM to about 100 mM Tris-HCl; about 0.1 mM to about 50 mM (NH₄)₂SO₄; about 0.1 to about 50 mM KCl; about 0.02 mM to about 10 mM MgSO₄; about 0.01 to about 0.5% Triton® X-100. In embodiments, the isothermal detection components have a pH of about 7.9 to about 14 at about 25° C. In an embodiment, the buffer can comprise: 20 mM Tris-HCl; 10 mM (NH₄)₂SO₄, 10 mM KCl; 2 mM MgSO₄; 0.1% Triton® X-100; and pH 8.8@25° C. It would be understood that other buffers having other compositions may also be used. In embodiments, the buffer can be ThermoPol® from New England Biolabs® or water. In embodiments, the buffer is water having an initial pH before heating of about 8.8.

In embodiments, the second temperature is about 25° C. to about 40°; about 30° C. to about 35° C.; about 30° C. to about 40° C.; about 35° C. to about 40° C.; or about 37° C. In embodiments, the second temperature is applied for an incubation period of about 30 seconds to about 60 minutes.

In some embodiments the second temperature can be about room temperature. The skilled artisan would understand that the incubation period will depend on the concentration of target of interest, in particular, the starting concentration.

The Cas enzyme can be a Cas12a enzyme. In embodiments, the Cas12a enzyme has an isoelectric point compatible with higher pH conditions used to keep the strands separated as temperature cools. In embodiments, the Cas enzyme can be a Cas12a enzyme with an isoelectric point from about 6.5 to about 9. In some embodiments, the Cas12a enzyme can be LbCas12a, AsCas12a, or ErCas12a or a combination thereof. In embodiments, the Cas12a enzyme has an isoelectric point of about 8 or higher.

In embodiments, the LbCas12a of the systems and methods of the present disclosure is derived from subspecies Lachnospiraceae sp. (for example, GenBank ID: MW477886.1). In embodiments, the AsCas12a of the systems and methods of the present disclosure is derived from subspecies Acidaminococcus sp. (for example, GenBank ID: MW477885.1). In embodiments, the ErCas12a of the systems and methods of the present disclosure is derived from subspecies Eubacterium rectale (for example, GenBank ID: MH347339.1).

In embodiments, the present disclosure also includes one-pot methods of detecting a target polynucleotide in a sample, for example, by combining the isothermal amplification/CRISPR/Cas system elements described above with a sample in a single reaction vessel, incubating the contents of the reaction vessel at a temperature of about 60-70° C. or higher (for example 85° C. or other temperature ranges as disclosure above) for a period of time and detecting the CRISPR-generated detectable signal or detectable molecule if the target polynucleotide is present in the sample. In embodiments, the contents of the reaction vessel are incubated at a temperature of about 60-65° C. (or other temperature ranges as disclosure above). In embodiments, the contents of the reaction vessel are incubated at a temperature of about 62-63° C. (or other temperature ranges as disclosure above). The period of time until a signal is detected can vary and may be from about 5 minutes to about 45 minutes. In embodiments, the contents of the reaction vessel are incubated for about 5-45 minutes, such as from about 10-30 minutes, about 15-30, minutes, and other intervening and overlapping ranges.

In embodiments of methods according to the present disclosure, methods can further include an isothermal amplification step. As described above, according to embodiments as described above, the elements (as described above) included in the reaction vessel with the sample can also include: one or more isothermal amplification components including, but not limited to, an isothermal amplification buffer and/or isothermal amplification enzymes and primers configured to recognize and amplify the target polynucleotide (for example via LAMP or RT-LAMP). Thus, in embodiments, the elements in the reaction vessel can include, a Cas12 CRISPR-associated (Cas) enzyme; one or more isothermal detection components, a CRISPR RNA (crRNA) sequence configured to bind to the target polynucleotide and a scaffold sequence configured to bind to the Cas enzyme to form a CRISPR/Cas complex upon binding of the crRNA sequence to the target polynucleotide; one or more isothermal amplification components, and a plurality of probes, each probe having an oligonucleotide element labeled with a detectable label, wherein the probe is configured to be cleaved by the Cas12a Cas enzyme when the guide sequence binds the target polynucleotide to generate a CRISPR-generated detectable signal or detectable molecule.

In some embodiments the target polynucleotide can be a DNA or RNA associated with a specific condition/disease, such as cancer, genetic disease, or infectious agent (e.g., bacteria, virus, fungal, etc.). In embodiments, the target polynucleotide is a virus, such as, but not limited to SARS-CoV-2, Human immunodeficiency virus (HIV), Hepatitis C virus (HCV), Trypanosoma cruzi, Plasmodium vivax, Plasmodium ovale (including subspecies Wallikeri and Curtisi), falciparum, malariae, knowlesi), bluetongue virus, zika virus, ebola virus, influenza virus, rhinovirus, ebola, and cancer targets including pancreatic cancer, prostate cancer, breast cancer, and bladder cancer targets such as mutated genes (GFR3, TP53, ERBB2, CDKN2A, KRAS, HRAS, MET, PIK3CA, MLL, TERT, and VHL) or overexpressed mRNAs (MDK, HOXA13, CDC2, IGFBP5, CXCR2, UPK1B, IGF2, CRH, ANXA10, and ABL1). Thus, in embodiments the crRNA can be complimentary to and configured to bind a complimentary region on the target DNA or RNA of interest to detect the specific condition/disease, such as cancer, genetic disease, infection agent (e.g., bacteria, virus, etc.) and the like.

According to the embodiments of methods of the present disclosure, when the sample is combined with all of these elements in the reaction vessel at the elevated temperatures, the target polynucleotide in the sample is denatured (for example from dsDNA to ssDNA) by the heat and/or isothermal detection components, and then the CrRNA can bind the amplified target polynucleotides and forms a CRISPR/Cas complex with the thermostable CAS12a (for example LbCas12a, AsCas12a, or ErCas12a) to activate the trans cleavage activity of the Cas12a (for example LbCas12a, AsCas12a, or ErCas12a), which cleaves probes in the reaction vessel to create a detectable signal and indicate the presence of the target polynucleotide in the sample, thereby providing information that can be utilized, for example, in the diagnosis the specific condition/disease associated with the target polynucleotide. Thus, in embodiments, the methods of the present disclosure can include diagnosing a condition associated with the target polynucleotide upon detection of the detectable signal.

According to the embodiments of methods of the present disclosure, when the sample containing a target is combined with all of these elements in the reaction vessel at the elevated temperatures, the target polynucleotide in the sample is amplified by the isothermal amplification components, and then the application of heat and the isothermal detection components (including the isothermal detection buffer) maintains the amplified target in single stranded form to allow the CrRNA to binds the amplified target polynucleotides (even in absence of PAM sequences) and forms a CRISPR/Cas complex with the Cas12a (for example LbCas12a, AsCas12a, or ErCas12a) to activate the trans cleavage activity of the Cas12a (for example LbCas12a, AsCas12a, or ErCas12a), which cleaves probes in the reaction vessel to create a detectable signal and indicate the presence of the target polynucleotide in the sample, thereby diagnosing the specific condition/disease associated with the target polynucleotide. Thus, in embodiments, the methods of the present disclosure can include diagnosing a condition associated with the target polynucleotide upon detection of the detectable signal.

In embodiments the target polynucleotide is from a virus such as, but not limited to, SARS-CoV-2, HIV, HCV, Chagas, malaria, bluetongue virus, etc., and the CRSPR/Cas12a complex binds the target polynucleotide, activating trans cleavage activity, cleaving the probes, and detecting the presence of the virus in the sample. In some embodiments the target polynucleotide is a specific for a variant of a virus, such as variants of SARS-CoV-2, and the specificity of the crRNA is specific enough to distinguish between variants of SARS-CoV-2 (such as, but not limited to Alpha (B.1.1.7), Beta (B.1.352), Delta (B.1.617.2), and Gamma (P1)), HCV (such as, but not limited to HCV-1a and HCV-1b), or HIV (such as, but not limited to HIVK103 and HIVN103) wherein the set of isothermal amplification components comprise loop-mediated isothermal amplification (LAMP) or reverse-transcription LAMP (RT-LAMP) enzymes and primers.

In some embodiments, the set of isothermal amplification components added to the reaction vessel also includes an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes. In some embodiments, the isothermal amplification primers (e.g., LAMP primers) are not as specific for different variants of a target polynucleotide (e.g., virus variants) and can amplify different variants of the same target (such as, different variants of a virus, including, for instance different variants of SARS-CoV-2, HIV, and/or HCV). Thus, in embodiments, the isothermal amplification reporter can detect the presence of a target polynucleotide associated with a specific condition, but may not be able to discern between different variants. However, the crRNA can be designed to specifically bind to a specific variant, such that the crRNA/Cas12a complex only binds and activates trans cleavage of the probes in the presence of the target polynucleotide from the specific variant. In embodiments, the detectable signal produced by the isothermal amplification reporter can indicate the presence of a target polynucleotide associated with a specific condition (e.g., SARS-CoV2, HIV, or HCV), and the CRISPR-generated detectable signal produced by cleavage of the probe indicates the presence of a specific variant of the condition. For instance, in an embodiment described in greater detail in the examples below, an isothermal amplification reporter can indicate the presence in the sample of any variant of SARS-CoV-2, HCV, or HIV, while cleavage of the probe by the CRISPR/Cas complex indicates the presence in the sample of a specific variant of SARS-CoV-2, e.g., Alpha (B.1.1.7), Beta (B.1.352), Delta (B.1.617.2), and Gamma (P1), or a specific variant of HIV (e.g., HIV-K103, HIV-N103, etc.) or specific variant of HCV (e.g., HCV-1a, HCV-1b, etc.). In embodiments the reaction vessel could contain different crRNAs each configured to specifically bind and identify a different variant of the target, allowing for detection of the condition/target as well as determination of the variant. This method has the advantage of allowing rapid, on-site, simultaneous determination of a positive condition in a patient as well as identification of circulating variants.

Additional description of embodiments of methods of the present disclosure are provided in the examples below. Other methods for use of the isothermal amplification/CRISPR/Cas systems of the present disclosure can be appraised by one of skill in the art.

### Kits

The present disclosure also includes kits including the system components described above to be combined with a sample and instructions for use. In embodiments, the kit is shelf-stable, such that it can be transported and stored at ambient temperatures until use. In embodiments, the contents of the kit are lyophilized, such that they are non-reactive and stable until combined with a sample and incubated at elevated temperatures for isothermal amplification and CRISPR/Cas detection.

In embodiments, kits of the present disclosure shelf-stable kit for detecting a target polynucleotide in a sample in a single-pot including the following components:
a) an isothermal detection buffer;
b) a lyophilized Cas12a CRISPR-associated (Cas) enzyme;
c) a lyophilized guide RNA sequence having a CRISPR RNA (crRNA) sequence configured to bind to the target polynucleotide and configured to interact with the Cas12a Cas enzyme to form a CRISPR/Cas complex upon binding of the crRNA sequence to the target polynucleotide;
d) a plurality of lyophilized probes, each probe having an oligonucleotide element labeled with a detectable label, where the probe is configured to be cleaved by the Cas12a Cas enzyme when the crRNA sequence binds the target polynucleotide to generate a detectable signal or molecule; and
instructions for combining components a-d in a single reaction vessel with a sample, incubating the sample at a first temperature of about 60-90° C. for a first period of time (other first temperature or first temperature range as disclosed herein), incubating the sample at a second temperature of about 25-40° C. for a first period of time and detecting the detectable signal or molecule.

In embodiments, kits of the present disclosure include a shelf-stable kit for detecting a target polynucleotide in a sample in a single-pot including the following components:
a) a set of lyophilized isothermal amplification components including isothermal amplification enzymes and primers configured to recognize and amplify the target polynucleotide;
b) an isothermal detection buffer;
c) a lyophilized Cas12a CRISPR-associated (Cas) enzyme;
d) a lyophilized guide RNA sequence having a CRISPR RNA (crRNA) sequence configured to bind to the target polynucleotide and configured to interact with the Cas12a Cas enzyme to form a CRISPR/Cas complex upon binding of the crRNA sequence to the target polynucleotide;
e) a plurality of lyophilized probes, each probe having an oligonucleotide element labeled with a detectable label, where the probe is configured to be cleaved by the Cas12a Cas enzyme when the crRNA sequence binds the target polynucleotide to generate a detectable signal or molecule; and
instructions for combining components a-e in a single reaction vessel with a sample, incubating the sample at a temperature of about 60-65° C. for a period of time, and detecting the detectable signal or molecule.

In embodiments, the kit also includes a lyophilized isothermal amplification buffer, such as a lyophilized version of NEB® LAMP Master Mix. In embodiments, the lyophilized isothermal amplification buffer does not contain glycerol. Kits of the present disclosure can also include any of the elements described in the systems and methods above. In embodiments, additional elements can also be lyophilized to render them shelf-stable.

In embodiments according to kits of the present disclosure, the isothermal detection component(s) can include an isothermal detection buffer. In embodiments, the buffer can be ThermoPol® from New England Biolabs® or water. Other suitable buffers may include other PCR buffers such as the standard Taq buffer, Q5 reaction buffer, and the Long Amp Taq buffer. In embodiments, the buffer is water having an initial pH before heating of about 8.8.

Additional details regarding the methods, systems, and kits of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

### VARIOUS ASPECTS AND EMBODIMENTS OF THE PRESENT DISCLOSURE

The present disclosure further includes the following aspects and embodiments.

Aspect 1. A method of detecting a target polynucleotide in a sample, the method comprising:
incubating the contents of a reaction vessel at a first temperature of about 60-90° C. for a first period of time, the reaction vessel comprising:
   a sample comprising one or more double-stranded target polynucleotides;
   one or more isothermal detection components comprising an isothermal detection buffer having a pH of about 7.9 or greater;
   a Cas12a CRISPR-associated (Cas) enzyme;
   an sgRNA sequence comprising a CRISPR RNA (crRNA) sequence configured to bind to a single strand of at least one of the one or more target polynucleotides and configured to interact with the Cas12a Cas enzyme to form a CRISPR/Cas complex upon binding of the crRNA sequence to the at least one target polynucleotide; and
   a plurality of probes, each probe comprising an oligonucleotide element labeled with a detectable label, wherein the probe is configured to be cleaved by the Cas12a Cas enzyme when the crRNA sequence binds the target polynucleotide to generate a CRISPR-generated detectable signal or detectable molecule;
incubating the contents of the reaction vessel at a second temperature of about 25-40° C. for a second period of time; and
detecting the CRISPR-generated detectable signal or detectable molecule if the target polynucleotide is present in the sample.

Aspect 2. The method of aspect 1, further comprising providing the sample in a single reaction vessel prior to incubating the vessel at a first temperature.

Aspect 3. The method of any one of aspects 1 or 2, wherein the isothermal detection buffer has a pH of about 8 to about 14 at room temperature or before heating to the first or second temperature.

Aspect 4. The method of any one of aspects 1 to 3, wherein the isothermal detection buffer has an overall salt concentration of about 10 mM to about 500 mM.

Aspect 5. The method of any one of aspects 1 to 4, wherein the isothermal detection buffer is water having a pH greater than 8 or ThermoPol®.

Aspect 6. The method of any one of aspects 1 to 5, wherein the Cas12a has an isoelectric point of about 6.5 to about 9.

Aspect 7. The method of any one of aspects 1 to 6, wherein the Cas12a is LbCas12a, AsCas12a, or ErCas12a.

Aspect 8. The method of any one of aspects 1 to 7, further comprising adding to the reaction vessel one or more isothermal amplification components comprising an isothermal amplification buffer compatible with the isothermal detection components and the Cas12a Cas enzyme.

Aspect 9. The method of aspect 8, wherein the isothermal amplification components further comprise an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

Aspect 10. The method of aspect 9, wherein the isothermal amplification reporter is an SYTO dye.

Aspect 11. The method of any one of aspects 1 to 10, wherein the probe is a FAM-polyT-Quencher or FAM-(T-rich)-Quencher (FAM-FQ) reporter.

Aspect 12. The method of any one of aspects 1 to 11, wherein the oligonucleotide element of the probe comprises a ssDNA and is about 80% or more of A and/or T.

Aspect 13. The method of any one of aspects 1-12, wherein the oligonucleotide element of the probe comprises a nucleotide sequence selected from TTATT and TTTTTTTT.

Aspect 14. The method of any one of aspects 1-13, wherein the first temperature is about 85° C.

Aspect 15. The method of any one of aspects 1 to 14, wherein the second temperature is about 37° C.

Aspect 16. The method of any one of aspects 1 to 15, wherein the first period of time is about 2 minutes to about 30 minutes.

Aspect 17. The method of anyone of aspects 1 to 16, wherein the second period of time is about 30 seconds to about 60 minutes.

Aspect 18. The method of any one of aspects 1 to 17, wherein the set of isothermal amplification components comprise loop-mediated isothermal amplification (LAMP) or reverse-transcription LAMP (RT-LAMP) enzymes and primers.

Aspect 19. The method of aspect 18, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

Aspect 20. The method of aspect 19, wherein the target polynucleotide is DNA and the set of isothermal amplification components comprise LAMP enzymes and primers and a thermostable polymerase.

Aspect 21. The method of aspect 19, wherein the target polynucleotide is RNA and the set of isothermal amplification components comprise RT-LAMP enzymes and primers and wherein the RT-LAMP enzymes comprise a thermostable reverse transcriptase and thermostable a polymerase.

Aspect 22. The method of any one of aspects M to M21, wherein the target polynucleotide is a SARS-CoV-2 polynucleotide, a hepatitis C virus (HCV) polynucleotide, a human immunodeficiency virus (HIV) polynucleotide, a malaria polynucleotide, or a bluetongue virus polynucleotide.

Aspect 23. The method of aspect 22, wherein the method can distinguish between variants of SARS-CoV-2, HCV or HIV.

Aspect 24. A one-pot nucleic acid detection system for detecting a target polynucleotide in a sample, the system comprising:
one or more isothermal detection components comprising:
   an isothermal detection buffer;
a Cas12a CRISPR-associated (Cas) enzyme;
an sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the Cas12a Cas enzyme when the guide sequence binds the target polynucleotide to generate a detectable signal or molecule.

Aspect 25. The system of aspect 24, further comprising a single reaction vessel configured to contain the elements of the system of aspect X in a single pot and further comprising a heating element to maintain the reaction vessel at a temperature of about 60-90° C. and 25-40° C.

Aspect 26. The system of any one of aspects 24 or 25, wherein the isothermal detection buffer has a pH of about 8 to about 14 at room temperature or before heating to the first or second temperature.

Aspect 27. The system of any one of aspects 24 to 26, wherein the isothermal detection buffer has an overall salt concentration of about 10 mM to about 500 mM.

Aspect 28. The system of any one of aspects 24 to 27, wherein the isothermal detection buffer is water having a pH greater than 8 or ThermoPol®.

Aspect 29. The system of any one of aspects 24 to 28, wherein the Cas12a enzyme has an isoelectric point of about 6.5 to about 9.

Aspect 30. The system of any one of aspects 24-29, wherein the Cas12a enzyme is LbCas12a, AsCas12a, or ErCas12a.

Aspect 31. The system of any one of aspects 24 to 30, further comprising one or more isothermal amplification components compatible with the isothermal detection components and the Cas12a Cas enzyme.

Aspect 32. The system of aspect 31, wherein the isothermal amplification components comprise an isothermal amplification buffer compatible with the isothermal amplification components and the Cas12a Cas enzyme.

Aspect 33. The system of aspect 32, wherein the isothermal amplification buffer is NEB LAMP Master Mix.

Aspect 34. The system of any of aspects 31 to 33, wherein the one or more isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

Aspect 35. The system of aspect 34, wherein the isothermal amplification reporter is SYTO9 dye and the probe is a HEX-FQ reporter.

Aspect 36. The system of any of aspects 31 to 35, wherein the one or more isothermal amplification components comprise loop-mediated isothermal amplification (LAMP) or reverse-transcription LAMP (RT-LAMP) enzymes and primers.

Aspect 37. The system of aspect 36, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

Aspect 38. The system of any aspects 31 to 37, wherein the target polynucleotide is RNA and the set of isothermal amplification components comprise RT-LAMP enzymes and primers and wherein the RT-LAMP enzymes comprise a thermostable reverse transcriptase and thermostable a polymerase.

Aspect 39. The system of any of aspects 31 to 38, wherein the target polynucleotide is a SARS-CoV-2 polynucleotide, a hepatitis C virus (HCV) polynucleotide, a human immunodeficiency virus (HIV) polynucleotide, a malaria polynucleotide, or a bluetongue virus polynucleotide.

Aspect 40. The system of aspect X14, wherein the system is configured to distinguish between variants of SARS-CoV-2, HCV or HIV.

Aspect 41. The system of any one of aspects 24-39, wherein the oligonucleotide element of the probe comprises a ssDNA and is about 80% or more of A and/or T.

Aspect 42. A shelf-stable kit for detecting a target polynucleotide in a sample comprising the following components:
a) an isothermal detection buffer;
b) a lyophilized Cas12a CRISPR-associated (Cas) enzyme;
c) a lyophilized sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide;
d) a plurality of lyophilized probes, each probe comprising an oligonucleotide element labeled with a detectable label, wherein the probe is configured to be cleaved by the Cas12a Cas enzyme when the crRNA sequence binds the target polynucleotide to generate a detectable signal or molecule; and
instructions for combining components a-d with a sample, incubating the sample at a first temperature for a first period of time, a second temperature for a second period of time, and detecting the detectable signal or molecule.

Aspect 43. The kit of aspect 42, wherein the isothermal detection buffer is lyophilized.

Aspect 44. The kit of aspect 42 or 43, wherein the kit further comprises one or more lyophilized isothermal amplification components comprising a lyophilized isothermal amplification buffer.

Aspect 45. The kit of any one of aspects 42 to 44, wherein the first temperature is about 60° C. to about 90° C.

Aspect 46. The kit of any one of aspects 42 to 45, wherein the first period of time is about 2 minutes to about 30 minutes.

Aspect 47. The kit of any one of aspects 42 to 46, wherein the second temperature is about 25° C. to about 40° C.

Aspect 48. The kit of any one of aspects 42 to 47, wherein the second period of time is about 30 seconds to about 60 minutes.

Aspect 49. The kit of any one of aspects 42 to 48, wherein the isothermal detection buffer is water having a pH of about 8 or greater at room temperature or before heating to the first or second temperature or ThermoPol®.

Aspect 50. The kit of any one of aspects 42 to 49, wherein the one or more lyophilized isothermal amplification components further comprises:
e) a set of lyophilized isothermal amplification enzymes and primers configured to recognize and amplify the target polynucleotide.

Aspect 60: The method, system, or kits of any of the foregoing aspects, where the target polynucleotide is specific for a variant of a virus, such as variants of SARS-CoV-2, HCV, HIV, and the like, and the specificity of the crRNA is specific enough to distinguish between the variants. For instance, the crRNA can be specific for variants of SARS-CoV-2, such as, but not limited to Alpha (B.1.1.7), Beta (B.1.352), Delta (B.1.617.2), and Gamma (P1); variants of HCV, such as but not limited to HCV-1a and HCV-1b; and variants of HIV, such as but not limited to HIV K103 (wild type) and HIV N103.

Aspect 61 includes methods, systems, or kits according to any of the foregoing aspects, where further comprising including in the reaction vessel an isothermal amplification reporter configured to produce an amplification-generated detectable signal or molecule upon amplification of the SARS-CoV-2, HCV, or HIV polynucleotide, wherein the detectable signal or molecule of the isothermal amplification reporter is different and distinguishable from the CRISPR-generated detectable signal or molecule produced by cleavage of the probes, wherein detecting the amplification generated detectable signal or molecule indicates the presence of SARS-CoV-2, HIV, or HCV in the sample, and detecting the CRISPR-generated detectable signal or molecule indicates the presence of the SARS-CoV-2, HIV, or HCV variant.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

PAM-Independent Detection of Nucleic Acids with PICNIC

### Introduction

The present example describes the development and testing of a PAM-independent method for detecting nucleic acids with CRISPR-Cas12a called PICNIC. Most Class 2 CRISPR/Cas systems, including CRISPR/Cas12 (Type V), CRISPR/Cas14 (Type V), and CRISPR/Cas 13 (Type VI) mediate a nonspecific collateral trans-cleavage of random DNA and RNA after binding and cis-cleavage of their target DNA or RNA. For CRISPR/Cas12a, this multiple-turnover trans-cleavage activity is only initiated once the crRNA/Cas12a complex is bound to its target ssDNA or dsDNA that acts as an activator. This trans-cleavage activity has been widely exploited for nucleic acid detection and has been combined with fluorescence-based, paper-based, and electrochemical-based sensing technologies to develop rapid and sensitive diagnostics.

However, current CRISPR/Cas12a systems are limited by their dependency on the protospacer adjacent motif (PAM) sequence. PAM is a short DNA sequence (usually 2-6 base pairs in length) that needs to be present on the DNA region targeted for cleavage by the CRISPR system. Without the PAM sequence, Cas enzymes do not bind and cleave double-stranded DNA sequences. Different Cas enzymes have widely varied PAM requirements. Cas9 requires a 5′-NGG-3′ PAM (where N is any base) whereas Cas12a prefers a 5′-TTTV-3′ PAM (where V is A, C, or G). The need for a PAM sequence limits the targeting ability of CRISPR and therefore hampers detection of nucleic acid sequences that have few or no PAM sequences present within them. Thus, engineering a CRISPR-based system that can function in a PAM-independent manner will have a huge utility in molecular diagnostics for a wide variety of targets.

Cas12a based CRISPR systems have been shown to only require a PAM sequence for dsDNA and not for ssDNA. Although ssDNA's can usually be detected in a PAM-independent manner, generation of ssDNA targets is challenging for some targets, as the ssDNA strands tend to anneal and re-form dsDNA molecules at the lower temperatures needed for detection using CRISPR/Cas systems. While some Cas enzymes have been used that can operate to some degree at higher temperatures such as used for isothermal amplification, where dsDNA can be maintained in single stranded conformation, these Cas enzymes are not readily available and can be expensive.

Thus, the present example demonstrates CRISPR/Cas detection systems capable of detection of PAMless dsDNA targets with the use of standard, readily available Cas12a enzymes. The present example describes development, testing, and optimization of PAMless Identification of NucleicAcids with CRISPR/Cas (PICNIC) methods and assays and use of the PICNIC methods and assays for detection of targets such as HCV and HIV and also distinction of single mutation variants thereof.

### Materials and Methods

### Plasmid Construction

Plasmids expressing Lb, As, and ErCas12a enzymes were constructed as described in Nguyen, Long T., et al., A Combinatorial Approach towards Adaptability of 22 Functional Cas12a Orthologs for Nucleic Acid Detection in Clinical Samples; 2021, https://www.medrxiv.org/content/I0.1101/2021.07.21.21260653v1, which is hereby incorporated herein in its entirety. Briefly, plasmids expressing LbCas12a and AsCas12a were obtained from Addgene (a gift from Zhang lab and Doudna lab) and directly used for protein expression. For ErCas12a, a plasmid containing the human codon optimized Cas12a gene was obtained from Addgene, then was PCR amplified using Q5 Hot Start high fidelity DNA polymerase (New England Biolabs, Catalog #M0493S), and subcloned into a bacterial expression vector (Addgene plasmid #29656, a gift from Scott Gradia). The product plasmids were then transformed into Rosetta™(DE3)pLysS Competent Cells (Millipore Sigma, Catalog #70956) following the manufacturer's protocols.

### Protein Expression and Purification:

For protein production, bacterial colonies containing the protein-expressing plasmid were plated on an agar plate and grown at 37° C. overnight. Individual colonies were then picked and inoculated for 12 hours in 10 mL of LB Broth (Fisher Scientific, Catalog #BP9723-500). The culture was subsequently scaled up to a 1.5 mL TB broth mix and grown until the culture reached an OD=0.6 to 0.8. The culture was then placed on ice before the addition of Isopropyl β-d-1-thiogalactopyranoside (IPTG) to a final concentration of 0.5 mM. The culture was then continued to grow overnight at 16° C. for 14-18 hours.

The overnight culture was pelleted by centrifuging at 10,000×g for 5 minutes. The cells were then resuspended in lysis buffer (500 mM NaCl, 50 mM Tris-HCl, pH=7.5, 20 mM Imidazole, 0.5 mM TCEP, 1 mM PMSF, 0.25 mg/mL Lysozyme, and DNase 1). The cell mixture was then subjected to sonication followed by centrifugation at 39800×g for 30 minutes. The cell lysate was filtered through a 0.22 μm syringe filter (Cytiva, Catalog #9913-2504) and then run through into 5 ml Histrap FF (Cytiva, Catalog #17525501, Ni2+ was stripped off and recharged with Co2+) pre-equilibrated with Wash Buffer A (500 mM NaCl, 50 mM Tris-HCl, pH=7.5, 20 mM imidazole, 0.5 mM TCEP) connected to BioLogic DuoFlow™ FPLC system (Bio-rad). The column was eluted with Elution Buffer B (500 mM NaCl, 50 mM Tris-HCl, pH=7.5, 250 mM imidazole, 0.5 mM TCEP). The eluted fractions were pooled together and transferred to a 10 kDa-14 kDa MWCO dialysis bag. Homemade TEV protease (plasmid was obtained as a gift from David Waugh, Addgene #8827, and purified in-house)(44) was added to the bag, submerged in Dialysis Buffer (500 mM NaCl, 50 mM HEPES, pH 7, 5 mM MgCl2, 2 mM DTT) and dialyzed at 4° C. overnight.

The protein mixture was taken out of the dialysis bag and concentrated down to around 10 mL using a 30 kDa MWCO Vivaspin® 20 concentrator. The concentrate was then equilibrated with 10 mL of Wash Buffer C (150 mM NaCl, 50 mM HEPES, pH=7, 0.5 mM TCEP) before injecting into 1 mL Hitrap Heparin HP column pre-equilibrated with Wash Buffer C operated in the BioLogic DuoFlow™ FPLC system (Bio-rad). The protein was eluted from the column by running a gradient flow rate that exchanges Wash Buffer C and Elution Buffer D (2000 mM NaCl, 50 mM HEPES, pH=7, 0.5 mM TCEP). Depending on how pure the protein samples were, additional size-exclusion chromatography may have been used. In short, the eluted protein from the previous step was run through a HiLoad® 16/600 Superdex® (Cytiva, Catalog #28989335). Eluted fractions with the highest protein purity were selected, pooled together, concentrated using a 30 kDa MWCO Vivaspin® 20 concentrator, snap-frozen in liquid nitrogen, and stored at −80° C. until use.

### Target DNA, RNA, and Guide Preparation:

All DNA and RNA oligos were obtained from Integrated DNA Technologies (IDT) unless otherwise specified. Single-stranded oligos were diluted in 1×TE buffer (10 mM Tris, 0.1 mM EDTA, pH 7.5). Complementary oligos for synthesizing dsDNA were first diluted in nuclease-free duplex buffer (30 mM HEPES, pH 7.5; 100 mM potassium acetate) and mixed in 1:1.5 molar ratio of target:non-target strand. Both strands were then subjected to denaturation at 95° C. for 4 mins and gradient cooling at a rate of 0.1° C./s to 25° C.

The PAM library consisting of 80 double-stranded DNA activators with varying PAM sequences was ordered from Twist Biosciences. These gene fragments were diluted in 1×TE buffer (10 mM Tris, 0.1 mM EDTA, pH 7.5) for long term storage. A working stock solution of 500 nM was diluted in water from the storage stock for using in experiments.

### CRISPR-Cas12a Reaction for Fluorescence-Based Detection:

All fluorescence-based detection assays were carried out in a low-volume, flat-bottom, black 384 well-plate. The crRNA-Cas12a conjugates were assembled by mixing them in NEB 2.1 buffer and nuclease-free water followed by incubation at room temperature for 10 min. The assembled crRNA-Cas12a mixes were then added to 500 nM FQ reporter and the appropriate concentration of the target activator in a 40-μl reaction volume. The 384 well-plate was then incubated in a BioTek Synergy fluorescence plate reader at 37° C. for 1 hour. Fluorescence intensity measurements for a FAM reporter were measured at the excitation/emission wavelengths of 483/20 nm and 530/20 nm every 2.5 min. A final concentration of 30 nM Cas12a, 60 nM crRNA, and 25 nM of target activator are used in all the assays unless otherwise specified.

### PICNIC Protocol

For performing PAMless detection with PICNIC, the target dsDNA activators were first subjected to denaturation at 85° C. in a highly basic environment (pH=12) for 10 min, or at pH and time period as described in the Example. The denatured activators were then subjected to the CRISPR-Cas based fluorescence detection protocol as described above.

### Recombinase Polymerase Amplification (RPA)

The RPA kit was purchased from Twist Biosciences (TwistAmp Basic Kit). For each reaction, 29.5 μL rehydration buffer, 2.4 μL of forward and reverse primer (10 μM), 3 μL target DNA, 10.2 μL nuclease free water and 2.5 μL MgOAc were combined together and incubated at 39° C. for 30 min. The amplified DNA products were then subjected to PICNIC denaturation and CRISPR based detection as described in the sections above.

### RESULTS & DISCUSSION

The present example demonstrates the superiority of the PICNIC methods and assays of the present disclosure over traditional CRISPR/Cas detection of targets that lack a canonical PAM sequence, particularly for detection of PAMless dsDNA targets. FIG. 1 is a schematic drawing illustrating the need of a protospacer adjacent motif (PAM) sequence for the detection of double-stranded DNA with traditional CRISPR-Cas12a. The trans-cleavage activity of Cas12a is active for a double-stranded DNA (dsDNA) sequence containing a PAM recognition site (top) but is OFF when PAM is absent (middle illustration). However, single-stranded DNA (ssDNA), even without a PAM site, is able to activate the trans-cleavage activity of Cas12a (bottom illustration). However, as discussed above, DNA does not tend to stay in single stranded form under normal assay conditions. To form ssDNA, the dsDNA target has to be heated to a temperature sufficiently high to denature the strands; however, upon cooling the strands tend to re-anneal to the double-stranded conformation. With most Cas enzymes, CRISPR/Cas detection cannot be performed at the high temperatures needed to maintain DNA in a single stranded conformation. The present example describes PICNIC methods and assays for detecting PAMless dsDNA targets with Cas12a enzymes.

The Schematic in FIG. 2 illustrates the core principle of the PICNIC methods and assays of the present disclosure. In PICNIC methods and assays, detection of a double-stranded DNA sequence without a canonical PAM recognition site is achieved by subjecting the dsDNA to a high temperature and high pH environment and subsequently separating the dsDNA into two separate ssDNA strands. The separated ssDNA strands can then be detected by Cas12a even in the absence of a PAM site even after cooling to a lower temperature.

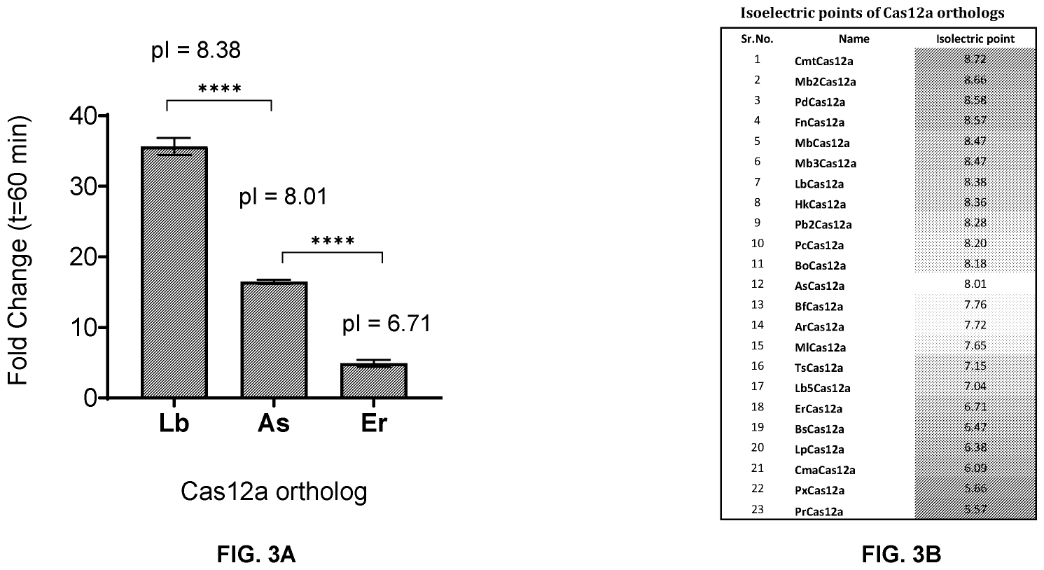

One principle of PICNIC is the use of a higher pH environment to maintain the DNA in single stranded conformation even after cooling slightly to temperatures conducive to detection with CRISPR/Cas systems. The graph in FIG. 3A characterizes the trans-cleavage activity of three different orthologs of Cas12a: LbCas12a, AsCas12a, and ErCas12a under high pH conditions (about pH 12). Data represents the fold change in the fluorescence intensity of a sample containing target dsDNA with respect to the no target control (NTC) at time t=60 min for n=3 replicates. The reaction contained 25 nM of target DNA, 30 nM crRNA, 60 nM Cas12a and 500 nM FAM-based reporter. Fluorescence measurements were done using a Synergy BioTek plate reader with excitation/emission wavelengths of 483/20 nm and 530/20 nm. Error bars represent SD (n=3). Statistical analysis was performed using a two tailed t-test where ns=not significant with p>0.05, and the asterisks (* p≤0.05, ** p≤0.01,*** p≤0.001, and **** p≤0.0001) denote significant differences. The Table in FIG. 3B displays the isoelectric points of 23 different Cas12a orthologs. All three orthologs tested worked with the methods and assays of the present disclosure, with increasing sensitivity of detection with increasing isoelectric point. Amongst the three orthologs tested in FIG. 3A, LbCas12a shows the best detection at high pH conditions likely because of a higher isoelectric point of Lb (pI=8.38) as compared to As (pI=8.01) or Er (pI=6.71).

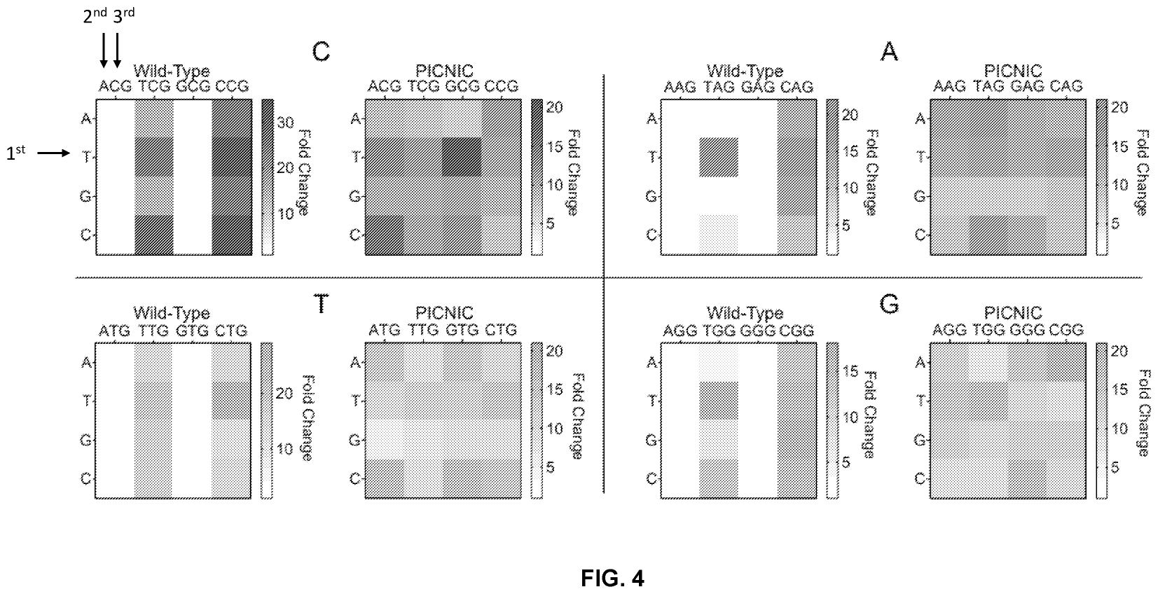

To demonstrate that the PICNIC assay allows detection of PAMless targets, a library of targets with different PAM sequences (canonical and non-canonical) was created and detection was tested according to standard (wild type) CRISPR/Cas assays and with the PICNIC methods/assays. While the experiments above demonstrate that the PICNIC assays methods can work with various Cas12a enzymes with isoelectric points, since higher activity was achieved with LbCas12a, unless otherwise specified, this Cas enzyme was used for the remainder of the experiments. FIG. 4 illustrates detection of a PAM library containing 64 unique PAM containing activators with wild-type CRISPR-Cas12a and PICNIC. The PAM library was designed by keeping the 4^{th} nucleotide of the PAM constant and varying the sequence of the first three nucleotides to contain all possible PAM combinations. The heat map represents the fold-change in the fluorescence intensity of the sample containing the target dsDNA with respect to the no target control (NTC) at time t=30 min for n=3 replicates. Both WT and PICNIC based detection was done using 25 nM of target DNA, 30 nM crRNA, 60 nM LbCas12a and 500 nM FAM-based reporter. For the PICNIC condition, the target dsDNA was heated at 85° C. for 10 min in a highly basic buffer (pH=12), before being subjected to CRISPR-based detection. For the WT condition, the target dsDNA was directly added to the CRISPR-reaction without any heating or pH adjustments. Fluorescence measurements were done using a Synergy BioTek plate reader with excitation/emission wavelengths of 483/20 nm and 530/20 nm respectively. While the WT CRISPR-Cas12a is able to detect T or C containing PAM sequences, it fails to detect A or G containing PAM activators. However, PICNIC is able to detect all 64 PAM sequences easily.

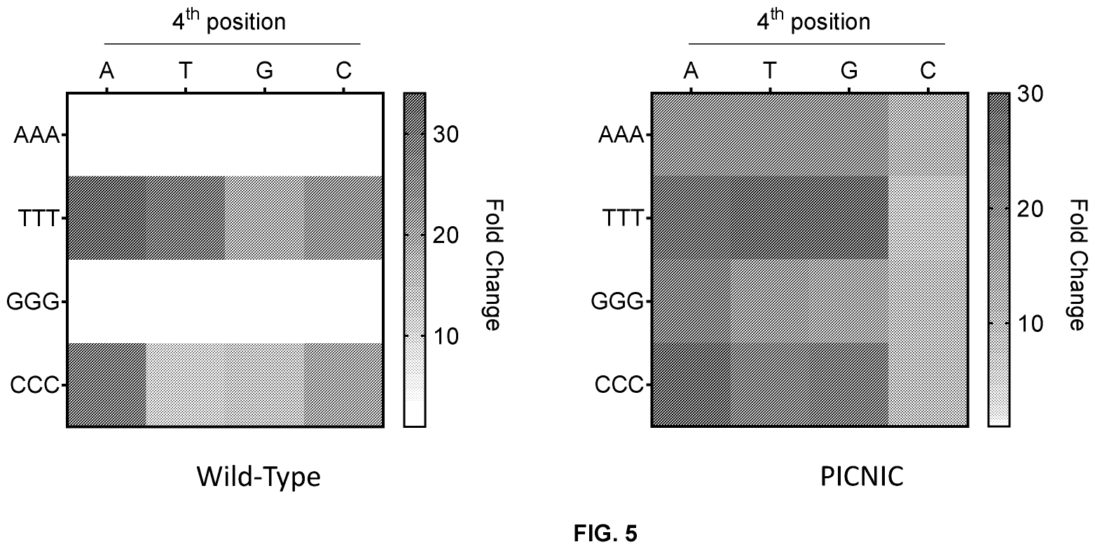

Similar to FIG. 4, FIG. 5 represents the detection of a PAM library containing 16 unique PAM containing targets. However, for this experiment, the first three nucleotides of the PAM were kept constant to be either AAA, TTT, GGG or CCC, and the 4^{th} nucleotide was varied to encompass all possible combination of sequences. The heat map images represent the fold-change in the fluorescence intensity of the sample containing the target dsDNA with respect to the no target control (NTC) at time t=30 min for n=3 replicates. Both WT and PICNIC based detection was done using 25 nM of target DNA, 30 nM crRNA, 60 nM LbCas12a and 500 nM FAM-based reporter. For the PICNIC condition, the target dsDNA was heated at 85° C. for 10 min in a highly basic buffer (pH=12), before being subjected to CRISPR-based detection. For the WT condition, the target dsDNA was directly added to the CRISPR-reaction without any heating or pH adjustments. Fluorescence measurements were done using a Synergy BioTek plate reader with excitation/emission wavelengths of 483/20 nm and 530/20 nm respectively. While the WT CRISPR-Cas12a is able to detect T or C containing PAM sequences, it fails to detect A or G containing PAM activators. However, PICNIC is able to detect all 16 PAM sequences easily.

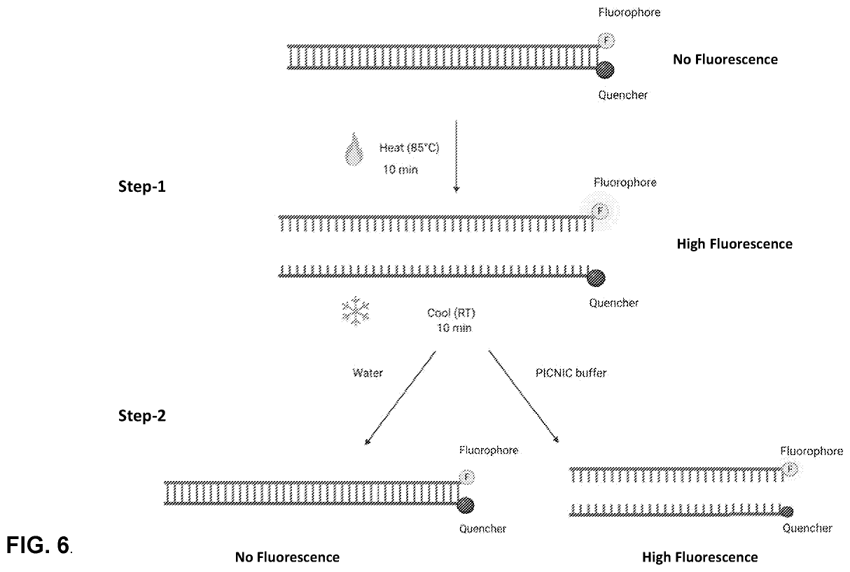

In order to test the hypothesis behind the PICNIC assay, experiments were designed to determine whether dsDNA was remaining in single stranded conformation after heating and cooling in the isothermal PICNIC detection buffer. FIG. 6 is a schematic illustration detailing the experiment performed to test the PICNIC hypothesis. To validate that the dsDNA is being denatured to separate ssDNA strands in PICNIC buffer, a synthetic dsDNA was designed to contain a fluorophore at the 3′-end of the top strand and a quencher at the 5′-end of the complementary strand. When the DNA is double-stranded, the fluorophore and quencher will be in close proximity to each other and there will be no fluorescence within the sample. However, when the strands denature to be separate ssDNA, the fluorophore will no longer be quenched, and the sample will fluoresce. To test if the dsDNA was being denatured to ssDNA in PICNIC buffer at high temperature and pH conditions, the labeled dsDNA was heated at 85° C. in either pH-neutral water (pH˜7) or PICNIC buffer (pH=12) for 10-min and cooled at room temperature. In water, after cooling, the dsDNA went back to being double-stranded and emits no fluorescence. However, in PICNIC buffer fluorescence is observed even after the sample is cooled down, confirming that the ssDNA strands stay separated in the high pH environment.

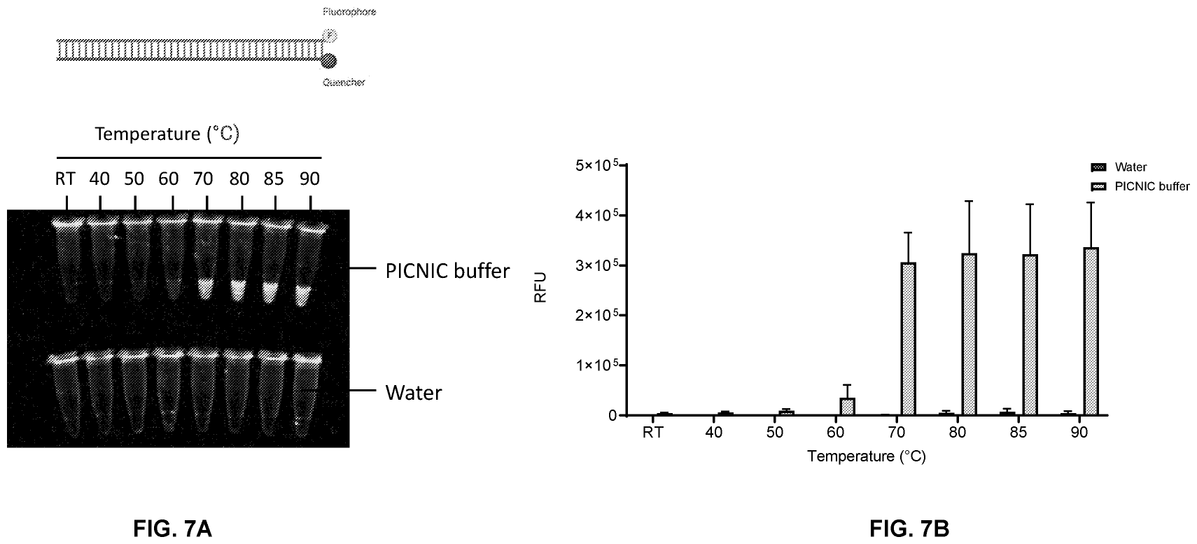

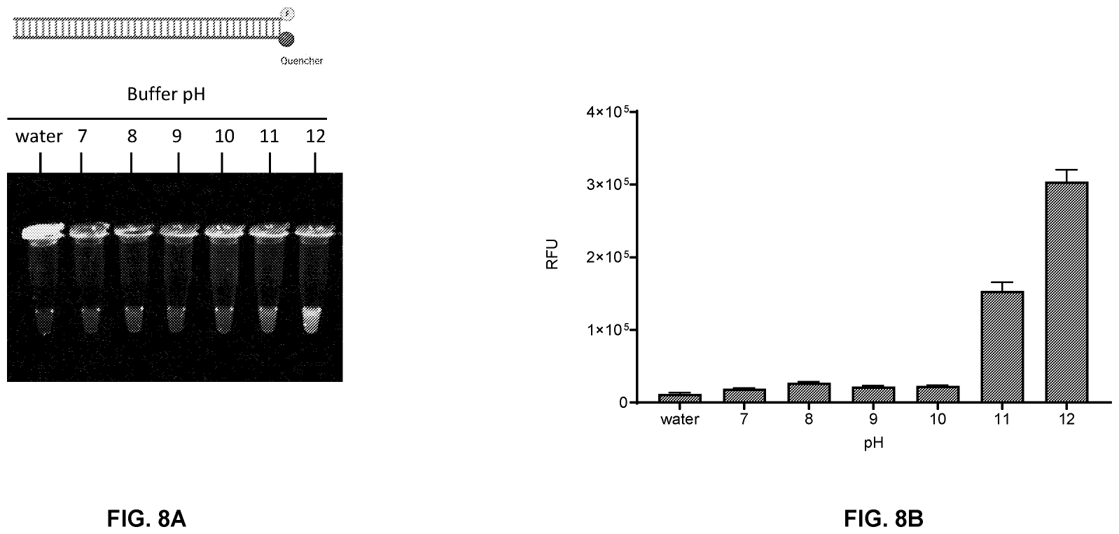

The results of the test illustrated in FIG. 6 are shown in FIGS. 7A-7B and FIGS. 8A-8B for testing the effect of various temperatures and pH's on the percentage of DNA in the double stranded vs. single stranded form. To test the effect of temperature on the denaturing ability of PICNIC, the labeled dsDNA was incubated at different temperatures ranging from Room Temperature (RT) to 90° C. for 10 min in either water (pH˜7) or PICNIC buffer (pH=12) and subsequently cooled down for 10 min. The fluorescence intensity in each sample was visualized under UV light using Analytik Jena UVP Gel Doc system (FIG. 7A). Fluorescence was observed only in the PICNIC buffer for incubation temperatures of about 70° C. or more, indicating that the heating step is important for separating the dsDNA with PICNIC. Three replicates were performed for this experiment, a representative image for one replicate is shown in FIG. 7A.

The fluorescence intensities of the samples in FIG. 7A were quantified using Synergy BioTek plate reader with excitation/emission wavelengths of 483/20 nm and 530/20 nm and illustrated in the graph form in FIG. 7B. Error bars represent SD (n=3). The fluorescence measurements corroborate the results from 7A and indicate that a heating step of 70° C. or more denatures the dsDNA in the PICNIC buffer.

FIGS. 8A-8B illustrate experiments performed to test the effect of pH on the denaturing ability of PICNIC. The labeled dsDNA was incubated for 10 min at 85° C. in different pH buffers ranging from pH=7 to pH=12 and subsequently cooled down for 10 min. The fluorescence intensity in each sample was visualized under UV light using Analytik Jena UVP Gel Doc system. An increase in the fluorescence intensity was observed with increasing pH of the buffer, confirming that a high pH facilitates denaturing the dsDNA into ssDNA and maintaining ssDNA after cooling using PICNIC. The fluorescence intensities of the samples in FIG. 8A were quantified using Synergy BioTek plate reader with excitation/emission wavelengths of 483/20 nm and 530/20 nm and presented graphically in FIG. 8B. Error bars represent SD (n=3). The fluorescence measurements corroborate the results from 8A and indicate that higher pH levels are better for denaturing dsDNA.

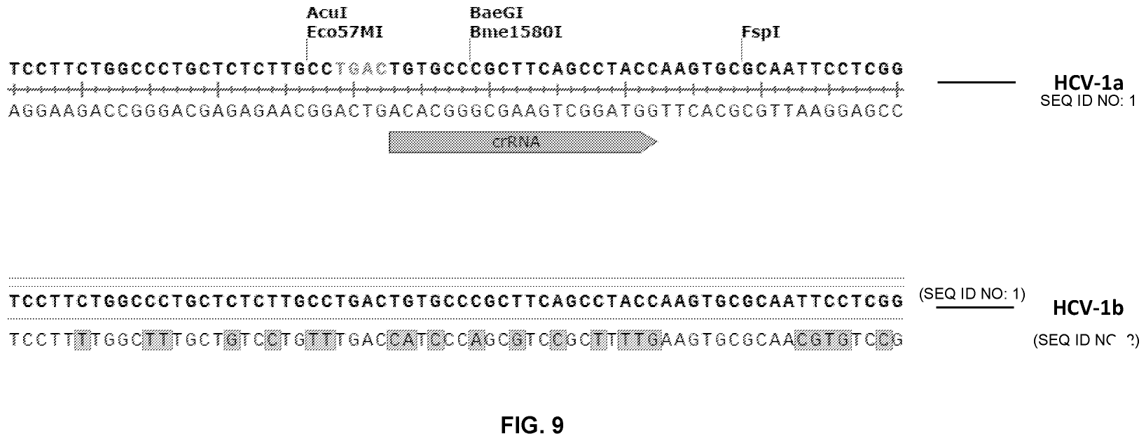

Hepatitis C virus (HCV) and variants were used as a model to demonstrate detection of a target sequence lacking a PAM. The schematic in FIG. 9 (top) shows a region of the genome of wild type HCV corresponding to the E1 gene (SEQ ID NO: 1) in dsDNA format. This is a hypervariable region in the HCV genome for genotypes 1a and 1b that can be targeted to discriminate between the two variants. FIG. 9 (bottom) shows a sequence alignment of HCV1a and HCV-1b showing various locations for point mutations. However, there are no canonical ‘TTTV’ PAM sites present near this sequence to perform CRISPR-based detection. crRNAs targeting the sequence adjacent to the ‘TGAC’ PAM was designed for both 1a and 1b. This region contains 10 mutations between HCV-1a and HCV-1b and can be used for genotyping. However, the lack of a canonical PAM site implies that traditional CRISPR-Cas systems can't target this region, only PICNIC will be able to do so.

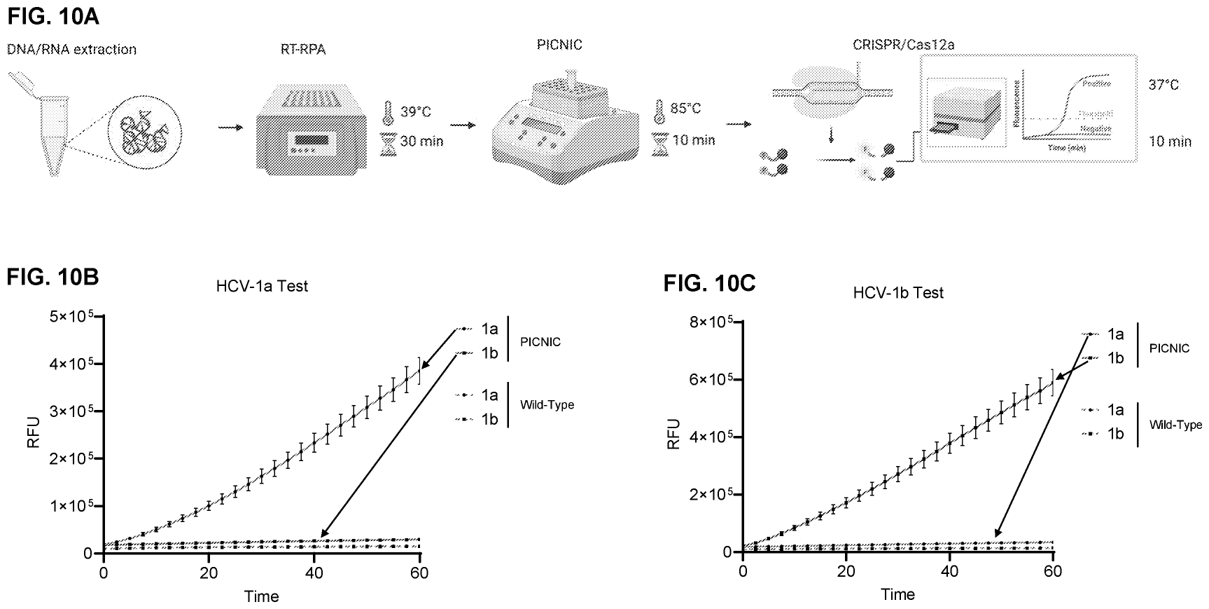

FIG. 10A is a schematic illustrating an embodiment of a workflow for HCV-genotyping with PICNIC. First, the clinical samples are subjected to nucleic acid extraction methods to purify the viral RNA from the complex sample matrix. The purified RNA is subjected to Reverse Transcription-Recombinase Polymerase Amplification (RT-RPA) at 39° C. for 30 min to generate dsDNA before being subjected to denaturation at high temperature and pH at 85° C. for 10 min in the high pH PICNIC buffer. The denatured DNA products are then subjected to fluorescence-based detection mediated by the trans-cleavage activity of CRISPR-LbCas12a at 37° C. for 30 min.

This method as used to compare detection and distinction of HCV1a from HCV1a using PICNIC vs. wild-type CRISPR detection. The plot in FIG. 10B represents the change in fluorescence intensity over time for the detection of HCV genotypes 1a and 1b with PICNIC and Wild-Type CRISPR-Cas systems with the test designed to detect HCV-1b. Synthetic DNA resembling the E1 gene in HCV genotypes 1a and 1b were first amplified with RPA before being subjected to CRISPR-based detection with the PICNIC protocol or just with wild type Cas12a. Notably, fluorescence is observed only in the 1a sample and only with the PICNIC assay. The crRNA designed for the HCV-1a test is specific to genotype-1a and therefore does not show trans-cleavage for the 1b target. Since the target is a non-canonical PAM containing site, fluorescence is observed only with the PICNIC protocol and not with the wild-type CRISPR-Cas12a. The plot in FIG. 10C represents the change in fluorescence intensity over time for the detection of HCV genotypes 1a and 1b with PICNIC and Wild-Type CRISPR-Cas systems with the test designed to detect 1HCV-1b. Synthetic DNA resembling the E1 gene in HCV genotypes 1a and 1b were first amplified with RPA before being subjected to CRISPR-based detection with the PICNIC protocol or just with wild type Cas12a. Notably, fluorescence is observed only in the 1b sample and only with PICNIC. The crRNA designed for the HCV-1b test is specific to genotype-1b and therefore does not show trans-cleavage for the 1b target. Like with FIG. 10B, since the target is a non-canonical PAM containing site, fluorescence is observed only with the PICNIC protocol and not with the wild-type CRISPR-Cas12a.

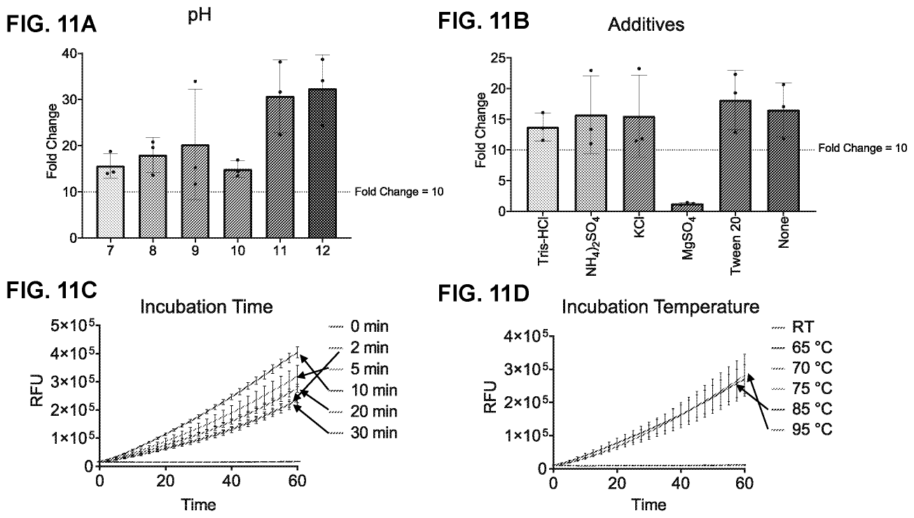

The PICNIC assay was optimized for pH, buffer additives, incubation time, and incubation temperature as illustrated in FIGS. 11A-11D. The plot in FIG. 11A represents fold change with respect to No Target Control (NTC) for different pH of the PICNIC buffer ranging from pH=7 to pH=12. The plot in FIG. 11B illustrates fold change with respect to No Target Control (NTC) for different additives such as Tris-HCl, ammonium sulfate, potassium chloride, magnesium sulfate and Tween-20 to the PICNIC buffer. FIG. 11C illustrates the increase in fluorescence intensity with time for different incubation time in the PICNIC step ranging from 0 min to 30 min. Finally, FIG. 11D graphs the increase in fluorescence intensity with time for different incubation temperatures in the PICNIC detection step ranging from room temperature (RT) to 95° C. For all of FIGS. 11A-11D, the reaction contained 25 nM of target DNA containing a non-canonical PAM sequence, 30 nM crRNA, 60 nM LbCas12a and 500 nM FAM-based reporter. Fluorescence measurements were done using a Synergy BioTek plate reader with excitation/emission wavelengths of 483/20 nm and 530/20 nm. Error bars represent SD (n=3).

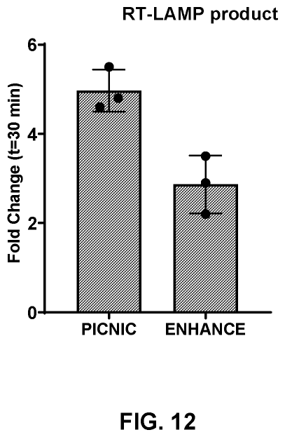

To demonstrate that PICNIC performs better trans-cleavage detection than ENHANCE with RT-LAMP product. A synthetic, PAMless dsDNA target was first amplified with RT-LAMP at 65° C. for 30 min and then subjected to detection with either CRISPR-ENHANCE or PICNIC (FIG. 12). The reaction contained 25 nM of target DNA containing a non-canonical PAM sequence, 30 nM crRNA, 60 nM Cas12a and 250 nM FAM-based reporter. The PICNIC assay was done by heating the RT-LAMP product at 85° C. for 10 min in Thermopol buffer. RNAse free water was used a template for the No Target Control (NTC). Fluorescence measurements were done using a Synergy BioTek plate reader with excitation/emission wavelengths of 483/20 nm and 530/20 nm. Error bars represent SD (n=3). The results demonstrate that the PICNIC assay is superior for detection off non-canonical PAM containing activators amplified with LAMP.

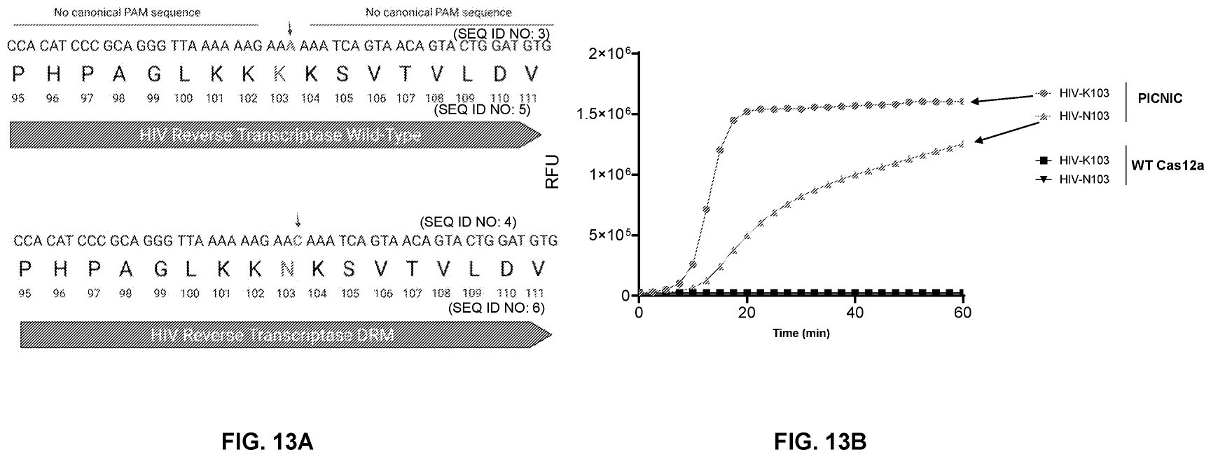

PICNIC was also used to test for detection of a single point mutation in the HIV genome in an area without a canonical PAM motif, where the mutation gives rise to resistance to HIV antiviral medications. FIG. 13A is a schematic showing a partial sequence within the reverse transcriptase gene of HIV. The Lysine (K) at position 103 is known to mutate to Asparagine (N) in some variants of HIV. The K103N variant makes the virus resistant to different non-nucleoside reverse transcriptase inhibitor drugs that are frequently administered to HIV patients. Thus, the K103N mutation makes the virus drug resistant and is therefore an important biomarker for detection. However, there are no canonical PAM sequence close to this mutation making it difficult to detect with traditional CRISPR-Cas12a based detection methods. However, PICNIC is able to detect these non-canonical PAM containing targets.

The graph in FIG. 13B represents the increase in fluorescence intensity with time for the detection of HIV K103 as well as the HIV N103 variant with wild-type CRISPR-Cas12a and PICNIC. Two synthetic DNAs resembling the gene fragments near HIV K103 and N103 mutation site were synthesized and subjected to detection with wild-type CRISPR and PICNIC. crRNAs were designed to detect both variants (K103, N103); however, the crRNAs targeted a non-canonical AAGA PAM site. Notably, only PICNIC was able to detect both targets, while WT CRISPR-Cas12a failed in the detection of both K103 and N103. The reaction contained 25 nM of target, 30 nM crRNA, 60 nM Cas12a and 500 nM FAM-based reporter. Fluorescence measurements were done using a Synergy BioTek plate reader with excitation/emission wavelengths of 483/20 nm and 530/20 nm.

SEQUENCES USED IN EXAMPLES

SEQ ID NO: 1-HCV-1a DNA
TCCTTCTGGCCCTGCTCTCTTGCCTGACTGTGCCCGCTTCAGCCTACCA
AGTGCGCAATTCCTCGG
SEQ ID NO: 2 HCV-1b DNA
TCCTTTTGGCTTTGCTGTCCTGTTTGACCATCCCAGCGTCCGCTTTTGA
AGTGCGCAACGTGTCCG
SEQ ID NO: 3 HIV Wild type (K103) DNA
CCACATCCCGCAGGGTTAAAAAAGAAAAAATCAGTAACAGTACTGGATG
TG
SEQ ID NO: 4 HIV variant N103, DNA
CCACATCCCGCAGGGTTAAAAAAGAACAAATCAGTAACAGTACTGGATG
TG
SEQ ID NO: 5 HIV K103 wt protein sequence:
PHPAGLKKKKSVTVLDV
SEQ ID NO: 6 HIV variant N103 protein sequence:
PHPAGLKKNKSVTVLDV

### REFERENCES

Tsou J H, Leng Q, Jiang F. A CRISPR Test for Rapidly and Sensitively Detecting Circulating EGFR Mutations. Diagnostics (Basel). 2020; 10(2):114. Published 2020 Feb. 19. doi:10.3390/diagnostics10020114
Tóth E, Varga É, Kulcsár P I, et al. Improved LbCas12a variants with altered PAM specificities further broaden the genome targeting range of Cas12a nucleases. Nucleic Acids Res. 2020; 48(7):3722-3733. doi:10.1093/nar/gkaa110
Nguyen, Long T., et al., A Combinatorial Approach towards Adaptability of 22 Functional Cas12a Orthologs for Nucleic Acid Detection in Clinical Samples; 2021, https://www.medrxiv.org/content/10.1101/2021.07.21.21260653v1
(Gootenberg et al., Science, 2017; Gootenberg et al., Science, 2018; Chen et al., Science, 2018; Brougton et al, Nat. Biotechnol., 2020; Young et al, NEJM., 2020

1. A method of detecting a target polynucleotide in a sample, the method comprising:
incubating the contents of a reaction vessel at a first temperature of about 60-90° C. for a first period of time, the reaction vessel comprising:
a sample comprising one or more double-stranded target polynucleotides;
one or more isothermal detection components comprising an isothermal detection buffer having a pH of about 7.9 or greater;
a Cas12a CRISPR-associated (Cas) enzyme;
an sgRNA sequence comprising a CRISPR RNA (crRNA) sequence configured to bind to a single strand of at least one of the one or more target polynucleotides and configured to interact with the Cas12a Cas enzyme to form a CRISPR/Cas complex upon binding of the crRNA sequence to the at least one target polynucleotide; and
a plurality of probes, each probe comprising an oligonucleotide element labeled with a detectable label, wherein the probe is configured to be cleaved by the Cas12a Cas enzyme when the crRNA sequence binds the target polynucleotide to generate a CRISPR-generated detectable signal or detectable molecule;
incubating the contents of the reaction vessel at a second temperature of about 25-40° C. for a second period of time; and
detecting the CRISPR-generated detectable signal or detectable molecule if the target polynucleotide is present in the sample.

2. The method of claim 1, further comprising providing the sample in a single reaction vessel prior to incubating the vessel at a first temperature.

3. The method of claim 1 wherein the isothermal detection buffer has a pH of about 8 to about 14 at room temperature or before heating to the first or second temperature.

4. The method of claim 1, wherein the isothermal detection buffer has an overall salt concentration of about 10 mM to about 500 mM.

5. The method of claim 1, wherein the isothermal detection buffer is water having a pH greater than 8 or ThermoPol®.

6. The method of claim 1, wherein the Cas12a has an isoelectric point of about 6.5 to about 9.

7. The method of claim 1, wherein the Cas12a is LbCas12a, AsCas12a, or ErCas12a.

8. The method of claim 1, further comprising adding to the reaction vessel one or more isothermal amplification components comprising an isothermal amplification buffer compatible with the isothermal detection components and the Cas12a Cas enzyme.

9. The method of claim 8, wherein the isothermal amplification components further comprise an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

10. The method of claim 9, wherein the isothermal amplification reporter is an SYTO dye.

11. The method of claim 1, wherein the probe is a FAM-polyT-Quencher or FAM-(T-rich)-Quencher (FAM-FQ) reporter.

12. The method of claim 1, wherein the oligonucleotide element of the probe comprises a ssDNA and is about 80% or more of A and/or T.

13. The method claim 1, wherein the oligonucleotide element of the probe comprises a nucleotide sequence selected from TTATT and TTTTTTTT.

14-23. (canceled)

24. A one-pot nucleic acid detection system for detecting a target polynucleotide in a sample, the system comprising:
one or more isothermal detection components comprising:
an isothermal detection buffer;
a Cas12a CRISPR-associated (Cas) enzyme;
an sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the Cas12a Cas enzyme when the guide sequence binds the target polynucleotide to generate a detectable signal or molecule.

25. The system of claim 24, further comprising a single reaction vessel configured to contain the elements of the system of claim 24 in a single pot and further comprising a heating element to maintain the reaction vessel at a temperature of about 60-90° C. and 25-40° C.

26-30. (canceled)

31. The system of claim 24, further comprising one or more isothermal amplification components compatible with the isothermal detection components and the Cas12a Cas enzyme.

32. The system of claim 31, wherein the isothermal amplification components comprise an isothermal amplification buffer compatible with the isothermal amplification components and the Cas12a Cas enzyme.

33. The system of claim 32, wherein the isothermal amplification buffer is NEB LAMP Master Mix.

34. The system of claim 31, wherein the one or more isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

35-41. (canceled)

42. A shelf-stable kit for detecting a target polynucleotide in a sample comprising the following components:
a) an isothermal detection buffer;
b) a lyophilized Cas12a CRISPR-associated (Cas) enzyme;
c) a lyophilized sgRNA sequence comprising a CRISPR RNA (crRNA) sequence configured to bind to a target polynucleotide and configured to interact with the Cas12a Cas enzyme to form a CRISPR/Cas complex upon binding of the crRNA sequence to the target polynucleotide;
d) a plurality of lyophilized probes, each probe comprising an oligonucleotide element labeled with a detectable label, wherein the probe is configured to be cleaved by the Cas12a Cas enzyme when the crRNA sequence binds the target polynucleotide to generate a detectable signal or molecule; and
instructions for combining components a-d with a sample, incubating the sample at a first temperature for a first period of time, a second temperature for a second period of time, and detecting the detectable signal or molecule.

43-50. (canceled)
